(19) United States
(12) Patent Application Publication
(10) Pub. No.: US 2025/0204947 A1
(43) Pub. Date: Jun. 26, 2025

(54) SURGICAL ACCESS DEVICE HAVING ONBOARD BALLOON VISUALIZATION MODULE

(71) Applicant: CardioSight, Inc., Dallas, TX (US)

(72) Inventors: Attila Meretei, Hanover, NH (US); Gergo Csikos, Budapest (HU); Attila Albini, Maglod (HU); Peter Csongvay, Budapest (HU); Kornel Horvath, Budaors (HU)

(73) Assignee: CardioSight, Inc., Dallas, TX (US)

(21) Appl. No.: 19/079,181

(22) Filed: Mar. 13, 2025

Related U.S. Application Data
(63) Continuation of application No. PCT/US2023/074407, filed on Sep. 15, 2023.
(60) Provisional application No. 63/482,000, filed on Jan. 27, 2023.
(60) Provisional application No. 63/384,372, filed on Nov. 18, 2022.
(60) Provisional application No. 63/376,049, filed on Sep. 16, 2022.

(51) Int. Cl. A61B 17/3205; A61B 1/00; A61B 1/05; A61B 17/34

(52) U.S. Cl. CPC A61B 17/32053; A61B 1/00082; A61B 1/05; A61B 17/3423

(57) ABSTRACT

A surgical access device having an onboard balloon visualization module is described. The balloon and visualization module may form part of a hybrid working channel when deployed. When stowed, the visualization module and balloon are enclosed completely within the elongate body of the surgical access device. Embodiments of the surgical access device and balloon visualization system may be employed in a variety of medical procedures.

### CROSS REFERENCE TO RELATED APPLICATIONS

This application is a continuation of International Patent Application No. PCT/US2023/074407, filed Sep. 15, 2023, which claims the benefit of U.S. Patent Provisional No. 63/376,049, filed Sep. 16, 2022, U.S. Patent Provisional No. 63/384,372, filed Nov. 18, 2022, and U.S. Patent Provisional No. 63/482,000, filed Jan. 27, 2023, each of which is incorporated by reference in its entirety herein.

### INCORPORATION BY REFERENCE

All publications and patent applications mentioned in this specification are herein incorporated by reference to the same extent as if each individual publication or patent application was specifically and individually indicated to be incorporated by reference.

### FIELD

The various alternative surgical access device embodiments relate to surgical devices with onboard imaging capabilities.

### BACKGROUND

Some conventional surgical devices provide visualization capabilities. However, one common shortcoming of the conventional designs is that the visualization system competes for the valuable real estate of the interior working channel of the device as shown in FIG. 1, which shows a view of a distal end of a conventional instrument. As such, there is a negative trade off to provide visualization systems at the expense of reductions in or compromises to working channel sizes. What is needed are improved visualization systems that allow increases in sizes for working channel while maintaining the desired surgical imaging capabilities.

### SUMMARY

In these and other embodiments, a surgical access device is provided. The device comprises an elongate body having a working channel; and a balloon visualization module positioned at the distal end of the elongate body, the balloon visualization module comprising a balloon with a camera module positioned within the balloon, wherein when in a stowed configuration, the camera module is configured to be contained within an outer diameter of the access sheath and when deployed in use with an inflated balloon, the camera module moves radially outwardly relative to a longitudinal axis of the elongate body to a position substantially beyond an outer wall of the elongate body.

In some embodiments, the balloon visualization module is completely within the working channel when stowed.

At least a portion of a distal surface of the balloon can form a contact visualization surface configured to contact target tissue and allow visualization of the target tissue therethrough by the camera module.

In some embodiments, at least 90% of the distal surface of the balloon is within a field of view of the camera module when the camera module is in a deployed configuration. In some embodiments, at least 95% of the distal surface of the balloon is within a field of view of the camera module when the camera module is in a deployed configuration.

A distal surface of the balloon can be rounded. In some embodiments, the balloon comprises an optically clear material.

The camera module can be spaced about 8-13 mm from a distal surface of the balloon.

In some embodiments, the elongate body comprises a plurality of lumens. The elongate body can comprise a fluid lumen. In some embodiments, a diameter of the fluid lumen is about 0.4-0.6 mm. The elongate body can comprise a position tracking sensor.

In some embodiments, the camera module comprises an electronic connectors extending proximally towards a proximal end of the elongate body. The camera module can comprise one or more lights. In some embodiments, the camera module is positioned generally parallel to a longitudinal axis of the elongate body. The camera module can be positioned generally parallel to a longitudinal axis of the elongate body. In some embodiments, the camera module is positioned at an angle relative to a longitudinal axis of the elongate body. The device can comprise a camera mount configured to attach to the channel extension and support the camera module.

The balloon visualization module can comprise a channel extension coupled to a distal end of the working channel. In some embodiments, the channel extension comprises a lumen with a cut out portion. The balloon, when inflated, can be configured to complete the lumen in at least part of the cut out portion. in some embodiments, the cutout portion comprises about 60-90% of a length of the channel extension. The channel extension can comprise a flexible tube. In some embodiments, the channel extension comprises an optically clear material.

The balloon can comprise a balloon channel configured to surround the channel extension. In some embodiments, a diameter of the balloon channel is about 3-3.8 mm. A length of the balloon channel can be about 16-17.4 mm. In some embodiments, the device comprises a camera mount configured to attach to the balloon channel and support the camera module. The balloon can comprise a thickness of less than or equal to about 0.1 mm. In some embodiments, the balloon comprises a durometer of about shore 80-90 A.

The balloon can be asymmetric around a longitudinal access of the channel extension. In some embodiments, more of a volume of the balloon is positioned above the channel extension. In some embodiments, greater than 80% of a volume of the balloon is positioned above the channel extension.

The elongate body can comprise diameter of about 12-16F.

In some embodiments, about 6-7 mm of a distal end of the channel extension is within a field of view of the camera module when the camera module is in a deployed position. In some embodiments, about 2-3 mm of a bottom portion of a distal end of the channel extension is within a field of view of the camera module when the camera module is in a deployed position. The channel extension can comprise about ⅓ of a bottom portion of a field of view of the camera module.

In some embodiments, a diameter of the working channel is about 8-9F. The working channel can extend past a distal end of the elongate body. In some embodiments, the balloon comprises one or a combination of polyurethane, silicone, Pebax®, Nylon, Polyester/PET. A proximal end of the balloon can be coupled to a distal end of the elongate body. In some embodiments, a height of the balloon is about 12-14 mm.

The camera module can comprise a field of view of about 110° in air. The camera module can comprise an integrated lighting system.

In some embodiments, a refraction index of a fluid within the balloon is substantially similar to a refraction index of the balloon.

The balloon can be responsive to a modulated pressure applied to the interior of the balloon. In some embodiments, when the balloon is responsive to a modulated pressure applied to the interior of the balloon the balloon is more compliant, less compliant, more firm, rigid, or the balloon deploys one or more of a feature, a surface or an auxiliary shape supported by the balloon.

A surface of the balloon can include one or more electrode, wiring, circuits or components for mapping or ablation applications performed using the surgical access device.

In these and other embodiments, a surgical access device is provided. The surgical access device comprises an elongate body having a working channel; and a balloon visualization module positioned at the distal end of the elongate body, the balloon visualization module comprising a balloon with a camera module positioned within an interior volume the balloon, spaced from a distal surface of the balloon, wherein when in a stowed configuration, the camera module is configured to be at least partially within a path of the working channel and when deployed in use with an inflated balloon, the camera module moves radially outwardly relative to a longitudinal axis of the elongate body out of the path of the working channel such that at least 90% of a distal surface of the balloon is within a field of view of the camera module.

In these and other embodiments, a method of performing a medical procedure using the surgical access device described above is provided. The device includes a proximal end having a handle or suitable coupling or configuration wherein the lighting, camera and/or visualization components of the balloon visualization module provide improved visualization capabilities as part of an arthroscopic, laparoscopic, endoscopic, robotically assisted or other surgical instrument used when performing the medical procedure.

In these and other embodiments, a method of performing a medical procedure on a patient is provided. The method comprises advancing a sheath into a desired position adjacent to or near a surgical site; moving a balloon visualization module out of the sheath; transitioning the balloon visualization module into a visualization state; positioning a distal portion of the sheath adjacent to the surgical site using imaging from a camera within the balloon visualization module; performing one or more steps of an interventional procedure using a tool delivered using the working channel and channel extension of the shaft; performing one or more steps of the interventional procedure under direct visualization of the surgical site, the tool or the surgical field using an output from the camera in the balloon visualization module; transitioning the balloon visualization module out of the visualization state; and returning the balloon visualization module into the stowed configuration within the sheath.

In some embodiments, the method comprises moving the BVM out of the sheath comprises advancing a shaft coupled to the BVM relative to the sheath. Transitioning the BVM into a visualization state can comprise inflating a balloon of the BVM with a fluid. Transitioning the BVM into a visualization state can comprise inflating a balloon of the BVM with saline.

In some embodiments, the method comprises purging fluid lines and/or a balloon of the BVM. Purging can comprise continuously pumping fluid through the fluid lines and balloon.

In some embodiments, transitioning the BVM into a visualization state comprises inflating a balloon of the BVM with a fluid. Inflating the balloon can comprise inflating until visual feedback confirms complete inflation. Inflating the balloon can comprise inflating until a predefined volume of fluid is pumped into the balloon. Inflating the balloon can comprise using active pressure control to provide one or more of constant balloon pressure, leak detection, and contact force sensing.

Transitioning the BVM into a visualization state can comprise inflating a balloon of the BVM with a fluid, thereby moving the camera into a desired position.

In some embodiments, the method comprises withdrawing the sheath and balloon visualization module from the patient.

The medical procedure can comprise ablation, left atrial appendage closure, minimally invasive mitral surgery, minimally invasive aortic surgery, coronary surgery, minimally invasive surgery for intrapericardial tumors, pacemaker lead removal, endomyocardial biopsy, transcatheter mitral valve repair, transcatheter mitral valve replacement, tricuspid valve repair, right ventricular reshaping, tether implantation, atrial septal defect or persistent foramen ovale closure, or balloon atrial septostomy.

In these and other embodiments, a method of performing a medical procedure on a patient is provided. The method comprises advancing a sheath into a desired position adjacent to or near a surgical site; moving a balloon visualization module out of the sheath; inflating a balloon of the balloon visualization module into a visualization state, thereby positioning a camera within the balloon visualization module into a desired position; positioning the balloon adjacent to the target tissue using imaging from a camera within the balloon visualization module; performing one or more steps of an interventional procedure using a tool delivered using the working channel and channel extension of the shaft; and performing one or more steps of the interventional procedure under direct visualization of the surgical site, the tool or the surgical field using an output from the camera in the balloon visualization module.

In these and other embodiments, a method of performing a medical procedure on a patient is provided. The method comprises navigating an access sheath to a desired location with a balloon visualization module in an uninflated stowed configuration within the access sheath; advancing the shaft comprising the BVM at its distal end out its stowed configuration within the access sheath; flushing fluid lines and a balloon of the BVM; pumping fluid into an interior volume of the balloon to initiating the transition from an uninflated to an inflated state; increasing the fluid volume, causing the balloon to unfold or unfurl and lifting the camera module of its stowed configuration; continuing to pump fluid into the interior volume of the balloon until it fully distends into an inflated state with the camera module in position for viewing the surgical field; and maintaining inflation of the balloon such that it gently complies with tissue at the treatment site during the procedure.

In some embodiments, the method comprises, upon completion of the procedure, deflating the balloon and retracting the BVM into the access sheath, causing the camera module to collapse of flex back into the outer diameter of the sheath. The method can comprise withdrawing the sheath and BVM. The fluid can be saline. In some embodiments, flushing fluid lines and the balloon comprises continuously pumping fluid through the fluid lines and balloon.

In some embodiments, continuing to pump fluid comprises pumping fluid until visual feedback confirms complete inflation. In some embodiments, continuing to pump fluid comprises pumping fluid until a predefined volume of fluid is pumped into the balloon. In some embodiments, continuing to pump fluid comprises using active pressure control to provide one or more of constant balloon pressure, leak detection, and contact force sensing.

The medical procedure can comprise ablation, left atrial appendage closure, minimally invasive mitral surgery, minimally invasive aortic surgery, coronary surgery, minimally invasive surgery for intrapericardial tumors, pacemaker lead removal, endomyocardial biopsy, transcatheter mitral valve repair, transcatheter mitral valve replacement, tricuspid valve repair, right ventricular reshaping, tether implantation, atrial septal defect or persistent foramen ovale closure, or balloon atrial septostomy.

In these and other embodiments, a method of performing a medical procedure on a patient is provided. The method comprises navigating an access sheath to a desired location with a balloon visualization module in an uninflated stowed configuration within the access sheath; advancing the shaft comprising the BVM at its distal end out of its stowed configuration within the access sheath; pumping fluid into an interior volume of the balloon to initiating the transition from an uninflated to an inflated state; and continuing to pump fluid into the interior volume of the balloon until it fully distends into an inflated state, placing a camera module within the balloon in position such that its field of view covers at least 90% of a distal face of the balloon.

In these and other embodiments, a method of performing a medical procedure is provided. The method comprises contacting tissue adjacent to a procedure site with a balloon of a balloon visualization module, displacing blood within the procedure site, wherein the balloon is inflated with a fluid; receiving imaging data from a camera module positioned within the balloon and the fluid; advancing a surgical tool through a working channel of the balloon visualization module, extending through the balloon; and visualizing the surgical tool as it exits the working channel using the camera module.

In these and other embodiments, a balloon for use with a surgical access device is provided. The balloon comprises an open proximal end; a proximal portion extending distally from the proximal end and comprising a generally tubular body; a midportion extending from the proximal portion, the midportion extending radially outwardly from the proximal portion along at least one of a top or bottom surface of the midportion and extending distally from the proximal portion to a mid-face section; a mid-face section extending radially inwardly from a distal end of the midportion forming a generally closed distal facing surface comprising an aperture; a distal portion extending from the aperture of the mid-face section, the distal portion comprising a generally tubular shape and an open distal end.

In some embodiments, when assembled for use with a surgical access device, the distal portion is inverted such that it is positioned within the midportion, forming a balloon channel within the midportion with the open distal end positioned proximal to the mid-face section. In some embodiments, when assembled for use with a surgical access device the open distal end is positioned within the midportion, proximal to the mid-face section, and distal to the open proximal end. In some embodiments, when assembled for use with a surgical access device, the mid-face section is the distal surface of the balloon. In some embodiments, when assembled for use with a surgical access device, the open proximal end of the balloon is coupled to a distal end of an elongate body.

The distal portion can be coupled to a lumen such that the lumen extends through the balloon channel. The lumen can comprise a flexible lumen. The lumen can comprise a rigid lumen with a cutout at its top portion. The lumen can comprise a rigid lumen. The lumen can comprise a lumen with a cutout at its top portion. In some embodiments, the cutout extends along about 60-90% of a length of the distal portion of the balloon. The lumen can comprise an optically clear material.

A camera module can be coupled to the balloon channel. In some embodiments, the camera module is coupled to the balloon channel such that a field of view of the camera module covers about 90% of a mid-face section of the balloon, when the balloon is inflated. The camera module can be coupled to the balloon channel using a camera mount.

In some embodiments, the open distal end is coupled to a distal end of a working channel of an elongate body. the distal portion can comprise a first durometer and the midportion and/or the mid-face section comprises a second durometer. The first durometer can be different from the second durometer.

In some embodiments, the open distal end is coupled to a distal end of a working channel of an elongate body. the distal portion can comprise a first flexibility and the midportion and/or the mid-face section comprises a second flexibility. The first flexibility can be different from the second flexibility.

In some embodiments, when assembled for use with a surgical access device, the distal portion is coupled to a lumen. The lumen can comprise a flexible lumen or a rigid lumen. The lumen can comprise a cutout top portion.

In some embodiments, the distal portion comprises a length of about 16-18 mm. The proximal portion can comprise a length of about 4-6 mm. In some embodiments, a diameter of the proximal portion is about 3-5 mm.

The proximal portion can be coaxial with the distal portion or not coaxial with the distal portion.

The balloon can be symmetrical or asymmetrical about a longitudinal axis of the distal portion. In some embodiments, a greater volume of the balloon is positioned above the distal portion than below the distal portion.

The mid-face section can be concave or convex.

The balloon can comprise an optically clear material. In some embodiments, the balloon comprises at least one of polyurethane, silicone, Pebax®, Nylon, and Polyester/PET.

### BRIEF DESCRIPTION OF THE DRAWINGS

FIG. 1 shows a perspective view of a distal end of a conventional instrument.

FIG. 2 shows a perspective view of a distal end of a surgical access device with a balloon visualization module (BVM).

FIGS. 3A and 3B show an embodiment of a surgical access device with a BVM being used in a procedure involving surgical access

FIG. 4A shows a side view of a distal end of an embodiment of an access device comprising a BVM.

FIG. 4B shows a representative view from a camera module.

FIG. 5A shows a side section view of a distal end of an embodiment of an access device comprising a BVM.

FIG. 5B shows a representative view from a camera module.

FIGS. 6A and 6B show side section and perspective views of a distal end of an embodiment of an access device comprising a BVM.

FIGS. 7A-7F show various views of a distal end of an embodiment of an access device comprising a BVM.

FIGS. 8A-8F show deployment of an embodiment of a BVM.

FIGS. 9A-9C show various views of an access device comprising a BVM in a stowed configuration.

FIG. 10 shows an isometric view of an embodiment of an access sheath and a BVM.

FIGS. 11A-11E show conventional pacemaker lead removal techniques.

FIG. 12 shows an embodiment of an access device comprising a BVM.

FIG. 13 shows a cross section side view of the device of FIG. 12 in position to remove a pacemaker lead.

FIGS. 14A-14C show various views of an embodiment of an access device comprising a BVM.

FIGS. 15A and 15B show section views of embodiments of multilumen shafts.

FIG. 16 shows a perspective view of a distal end of an embodiment of an extension channel.

FIGS. 17A and 17B show side section views of a balloon and a BVM comprising the balloon.

FIGS. 18A and 18B show various configurations of camera module mounts.

FIG. 19A shows a side view of a BVM.

FIG. 19B shows a representative view from the camera module of BVM.

FIGS. 20A and 20B show perspective views of an embodiment of a balloon during two steps of the assembly process.

FIGS. 21A-21D show side views of various embodiments of a balloon for use with a BVM.

FIGS. 22A-22C show various views of embodiments of methods for connecting a balloon with a channel extension.

FIGS. 23A-23D show various views of embodiments of methods for connecting a camera support to a distal leg of a balloon.

FIGS. 24A-24D show embodiments of a method for connecting a shaft to a balloon and channel extension.

FIG. 25 shows an embodiment of a proximal end of a BVM device.

FIGS. 26A and 26B show various views of an embodiment of a steerable BVM device.

FIGS. 27A and 27B show embodiments of methods for balloon inflation and deflation.

FIG. 28 shows an embodiment of a channel extension.

FIGS. 29A-29K show various embodiments of channel extensions comprising various proximal and distal mating features.

FIGS. 29L and 29M show perspective and side views of an embodiment of a channel extension.

FIGS. 29N and 290 show perspective and side views of a channel extension.

FIGS. 30A-30D show various views of embodiments of channel extensions comprising multiple lumens.

FIGS. 31A1-31M2 shows various embodiments and configurations for a number of different possible channel extension s comprising various cutout portions and proximal and distal mating feature configurations.

FIGS. 32A-32C show an embodiment of a method for deploying a BVM from a stowed configuration.

FIG. 33 shows an embodiment of a BVM comprising multiple camera modules.

FIG. 34 shows an embodiment of an access device with a BVM.

FIG. 35 shows an embodiment of a method of using a BVM device.

FIG. 36 shows an embodiment of a method of using a BVM device.

FIGS. 37A-37D show embodiments of balloons comprising distal windows with optical scales.

FIG. 38A shows a field of view from a camera module as shown in FIGS. 37A and 37C.

FIG. 38B shows a field of view from a camera module as shown in FIGS. 37B and 37D.

FIG. 39 shows an exemplary method of using a BVM as a tissue manipulator.

FIG. 40A shows a field of view from the camera module during an ablation procedure.

FIG. 40B shows a BVM device positioned towards the pulmonary veins.

FIG. 41A shows the field of view from a camera module during a pacemaker lead removal.

FIG. 41B shows a BVM device visualizing a scarred pacemaker lead site.

FIG. 42A shows a field of view from the camera module of a BVM device during a pacemaker lead removal with a pacemaker lead within the working channel.

FIG. 42B shows a circular cutting element delivered through the working channel.

FIG. 42C shows a view of the pacemaker lead inserted into the working channel of a BVM device and serving as a guide wire.

FIG. 43A shows a view from a camera module of BVM device performing a biopsy procedure at an annulus of a valve.

FIG. 43B shows the BVM device positioned toward the target tissue.

FIGS. 44A-44D show various embodiments of camera supports or mounts.

FIGS. 45A-45C show various embodiments of camera supports or mounts.

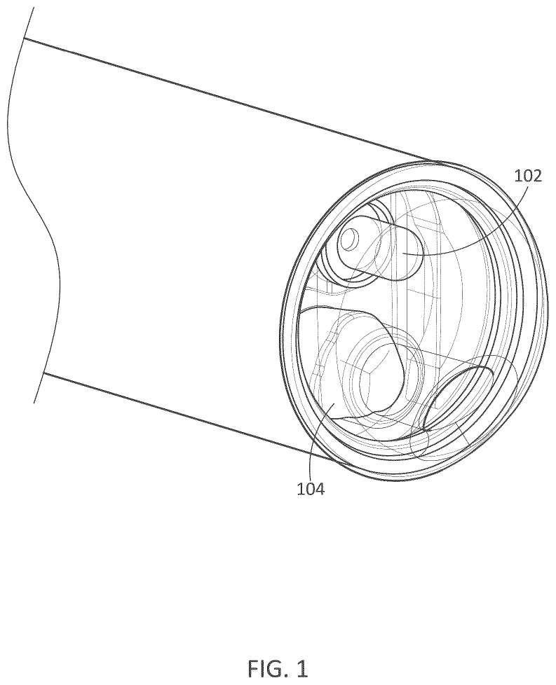

### DETAILED DESCRIPTION

As described herein, the various alternative embodiments of the inventive surgical access device with a balloon based visualization module (BVM) may be used to address a wide range of clinical issues in a number of different medical procedures.

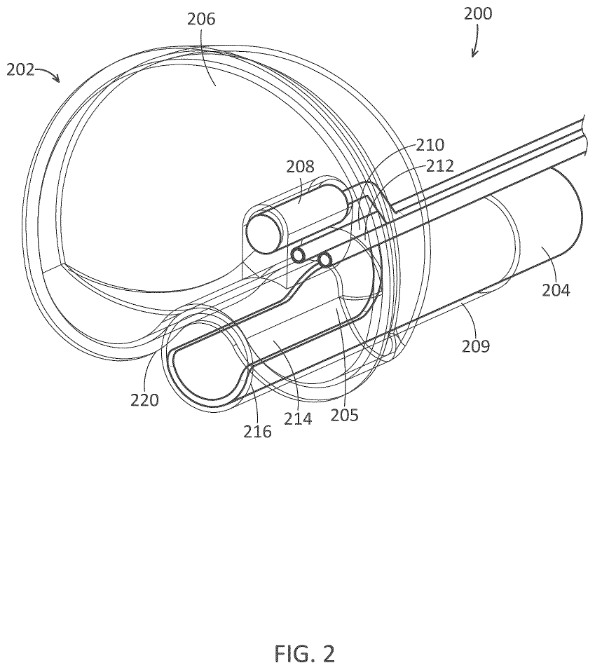

Referring now to FIG. 2, a perspective view of a distal end of a surgical access device 200 with a BVM 202 is shown. The device 200 comprises a shaft 204. A working channel 205 extends through the shaft 204. Positioned at or near a distal end of the shaft 204 is a balloon 206. Positioned within the balloon 206 is a camera and lighting system 208.

An inflow channel 210 and an outflow channel 212 are in fluid communication with the balloon 206. The inflow channel 210 can be used to inflate the balloon with fluid (e.g., saline, water, etc.). The outflow channel 212 can be used to evacuate bubbles from the balloon during inflation.

A distal end of the shaft 204 comprises a transparent portion 209. In some embodiments, the transparent portion 209 comprises an extension portion 216 and a cutout portion 214 through which the BVM is configured to at least partially emerge upon inflation of the balloon.

FIG. 2 shows the balloon comprises a shape resembling a truncated portion of a sphere positioned above the working shaft. The sphere is truncated at a distal end of the balloon to create the optical window. The sphere is truncated at a bottom end of the balloon around the shaft and/or working channel. A bottom central portion of the balloon comprises a recessed portion 220. In some embodiments, a shape and size of the recessed portion 220 can be configured to correspond to a size of a distal end of the working channel of the shaft. In some embodiments, other balloon shapes are used. Alternative balloon shapes can be selected to provide a range of different field of view coordination with a selected imaging/lighting components. Some aspects of the balloon shape and dimensions correspond both to the physical exterior size and shape of the imaging components as well as the optics associated with the functionality of the imaging components.

The balloon can comprise silicone. Other materials are also contemplated.

The balloon can comprise a thickness of about 0.20-0.25 mm (or about 0.15-0.30 mm, about 0.10-0.35 mm, about 0.05-0.1, 0.075-0.1, about 0.1 mm etc.)

Advantageously, in the stowed configuration in which the balloon 206 is deflated, the BVM is positioned within the extension portion 216 at the distal end of the shaft 204. The BVM can maintain this position during insertion and advancement to the surgical site. In contrast to conventional systems, the BVM components are placed into the cross section of the working channel so a considerably smaller outer diameter can be achieved. Upon balloon inflation, balloon and camera/lighting system move out of the way beyond the size of the outer diameter of the shaft 204. When the balloon is fully or mostly inflated, the camera 208 is in the desired position and the balloon provides the desired field of view.

In some embodiments, a bottom portion or profile 218 of the balloon, when inflated, forms a portion of the working channel in or around the cutout portion. In some embodiments, the bottom profile of the balloon is a portion of the working channel of the shaft. In some embodiments, a bottom portion of the balloon comprises a feature that forms a distal most part of the working channel.

In some embodiments, the extension portion 216 resembles a half or partial pipe shape, forming a portion of a tube shape. Other configurations are also contemplated. For example, in some embodiments, the extension portion 216 comprises a round and flexible extension at a distal end of the shaft 204 (e.g., as shown with respect to device 600, 700, 800, 900, etc.).

In some embodiments, the device comprises an outer diameter of about 3-10 mm, or about 4-8 mm, or about 5-7 mm, etc. Accordingly, it is to be appreciated that in various embodiments the device shaft, working channel and balloon visualization module are adapted and configured for use in performing one or more steps of a minimally invasive, single port access, robotically assisted, interventional or surgical procedure to address the shortcomings of or clinical issues related to a variety of medical procedures. In some embodiments, the surgical access device elongate body may be adapted and configured along all or a section of its length to be flexible, bendable or controllable into a range of different shapes or curvatures in support of the advantageous positioning and use of the balloon visualization module and associated medical procedures. For example, the surgical access device can be used with a sheath configured for steerability (e.g., 60-90° steerability in one direction). The handle or proximal end would be adapted and configured to allow user or robotic control of such functionality of the elongate body.

In general, embodiments of the inventive surgical access device having an onboard balloon visualization module includes a proximal end having a handle or suitable coupling or configuration wherein the lighting, camera and/or visualization components of the balloon visualization module provide improved visualization capabilities as part of an arthroscopic, laparoscopic, endoscopic, robotically assisted or other surgical instrument. The surgical access device also has an elongate body with an interior lumen. The interior lumen of the elongate body is used for a working channel extending from the proximal end to the distal end of the elongate body. The proximal end in communication with the handle and the distal end coupled to or in communication with the bvm support structure. The internal lumen of the elongate body also provides access for an inlet and an outlet to the balloon interior volume (i.e., to control inflation with fluid as well as manage bubbles and deflation or other functions). Additionally, the interior lumen of the elongate body is used for input, output, power, fiber optic and any other connections as are common in surgical vision systems depending upon the type of imaging desired based on the medical procedure being performed. These various additional connections are also within the lumen of the elongate body. As described further herein, the balloon visualization module is coupled to the distal end of the elongate body. There is a support structure that extends from the distal end of the elongate body. The balloon and visualization components when deflated/stowed rest on and along the support structure. When the balloon is deployed/inflated, the balloon is above the support structure and a portion of the balloon surface and the support structure further extends the working channel. Additionally or optionally, the balloon may be compliant and operable to confirm to the surgical field, have a rounded, circular or cylindrical outer overall shape or other external shape to accommodate imaging of complex geometry or in order to further assist in moving blood from the surgical field of view. These shape and visual field design aspects may correspond to various medical procedures described herein. The elongated body can be configured for use with an access sheath. The access sheath can be steerable or otherwise controllable.

In particular, in certain embodiments, the balloon visualization module (bvm) is advantageously configured in order to provide a portion of a hybrid working channel, a visualization chamber related to a desired field of view for an imaging and lighting system or component along with capability to transition from a stowed to a deployed configuration. The hybrid working channel refers to the advantageous combination of the working channel that is within the shaft of the access device and the working channel that is extended from the distal end of the access device. The extension of the working channel is the combination of the partial or half pipe base piece and the portion of the balloon bottom surface or contour that forms the upper portion of the working channel. In some embodiments, the hybrid working channel is the same inner diameter along the entire length, or the inner diameters may be different with one smaller than the other.

The balloon visualization module (BVM) is attached at the proximal end to the surgical access tool shaft. The balloon used in the balloon visualization module has a shape and size as to the distal face which is similar to a screen so as to mimic a desired field of view for the on board camera/lighting components associated with the balloon visualization module. The balloon visualization module can have a bottom surface that lays along, in, or along the half pipe extension from the distal end of the device. The half pipe or partial pipe extension is in alignment with and considered an extension of the working channel lumen of the surgical access device. While illustrated along the bottom, it is to be appreciated that the BVM extension may in other relationships relative to the working channel as in aligning on the top or sides or at any intermediate peripheral position (e.g., like the various angular positions on a clock face.). Additionally or optionally, a bottom surface of the inflated balloon may be used to form a distendable upper wall of the working channel. In some embodiments, the balloon comprises a channel configured to surround the extension of the working channel. In some embodiments, the stowed/deflated condition the balloon visualization module blocks completely or partially the working channel of the surgical access tool. In some embodiments, even in the stowed condition, the bottom surface of the balloon is above and creates an upper side of the hybrid working channel.

As a result, in some embodiments, the working channel of the BVM surgical access device is comprised of the composite of the working channel within the elongate body lumen along with the passage formed by the balloon extended or half pipe extender, optionally, along with some portion or a surface of the inflated/deployed balloon.

In additional or optionally, in some embodiments the various components and parts of the surgical access device such as the handle, the elongate body, the working channel, the balloon and the visualization module including balloon distal shape/image and lighting field of view are adapted and configured to provide desired access and imaging capabilities for surgical, interventional, endoscopic, arthroscopic, minimally invasive, single port access, open, general or robotic assisted medical procedures as described herein.

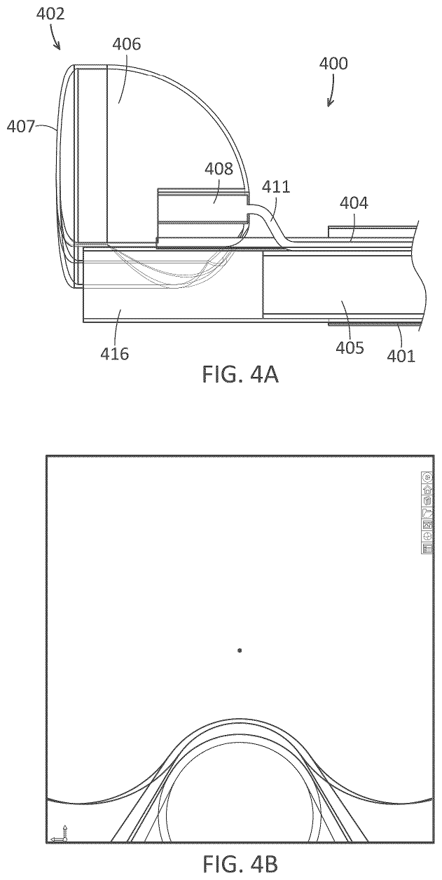

Referring now to FIG. 4A, a side view of a distal end of an embodiment of an access device 400 comprising a BVM 402 is shown. The device 400 comprises a shaft 404 comprising a working channel 405 extending therethrough. An optically clear window 407 is positioned at a distal end of the balloon 406. In some embodiments, the entire balloon is optically clear. In some embodiments, a distal portion of the balloon 406 is optically clear.

The cameral module 408 is positioned within the balloon 408. A connector, wire or cable 411 can extend from the camera module 408 to a proximal end of the device and can be used to provide data and/or power to the camera module.

In some embodiments, the distal window 407 of the balloon comprises a diameter of about 10-15 mm, and provides a field of view with a diameter of about 10-15 mm.

In some embodiments, a diameter of the shaft is about 5-6 mm. Other configurations are also contemplated (e.g., 3-8 mm, 4-7 mm, etc.).

In some embodiments, a diameter of the working channel is about 3-5 mm (or about 3 mm, 4 mm, 5 mm, etc.).

In some embodiments, the distal window comprises a diameter about 2 times the size of the shaft diameter (or about 3 times, or about 2-3 times, etc.). In some embodiments, the distal window comprises a diameter of about 3-5 times a diameter of the working channel (or about 3 times, or about 4 times, or about 5 times, etc.).

Moving to FIG. 4B, a representative view from the camera module 408 is shown. A transparent distal portion of the working channel 405 and an interior surface 411 of the balloon are visible in the field of view. A device extending through the working channel 405 would be within the field of view.

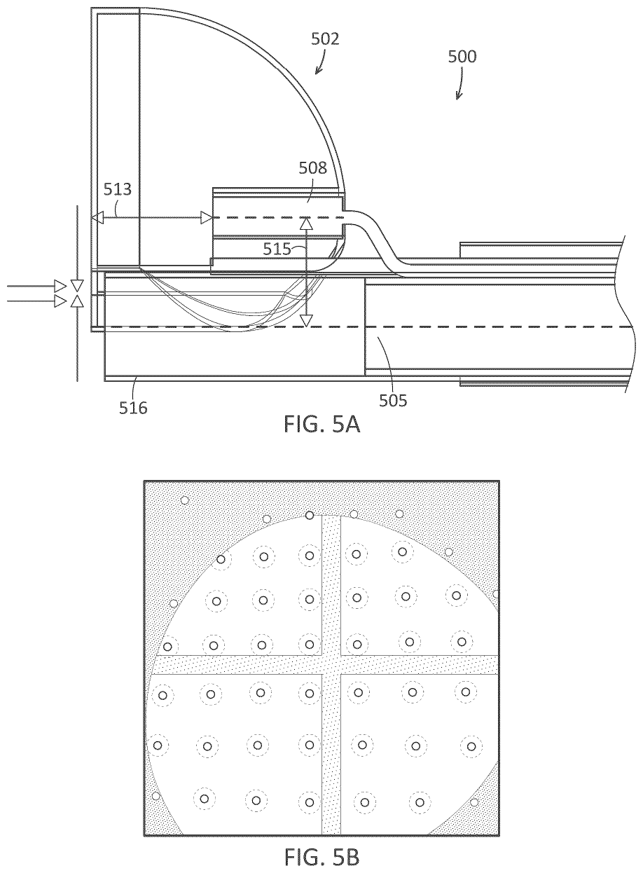

Referring to FIG. 5A, a view of an embodiment of an access device 500 with a BVM 502 positioned at its distal end is shown.

In some embodiments, as shown in FIG. 5A, the balloon 506 comprises a silicone balloon. A thickness of the balloon is about 0.25 mm.

As shown in FIG. 5A, the camera module can 508 comprise a diameter of about 1.6 mm. A length of the camera module 508 can be about 5 mm. In some embodiments, the camera module comprises an integrated lighting system (e.g., LED lighting). The camera module can comprise a view angle of about 110°.

In some embodiments, as shown in FIG. 5A, the camera module is set back from the distal window 507 of the balloon 506 by a distance 513. In some embodiments, the distance 513 is about 8 mm (or about 7-9 mm, about 7 mm, about 9 mm, etc.).

In some embodiments, a longitudinal axis of the camera module is positioned above a longitudinal axis of the shaft 504 and/or working channel 505 by a distance 515. In some embodiments, the distance 515 is about 3.5 mm (or about 3-4 mm, about 3 mm, about 4 mm, etc.).

As described herein, the access devices with BVM described herein can be adapted and configured to provide a working channel for use with instruments and/or various implants. The instruments can include but are not limited to a grasper, a snare, a biopsy needle, an RF ablation device, a tissue macerator, and an ultrasound probe.

FIG. 5B shows a field of view from camera 508 in a simulated use environment.

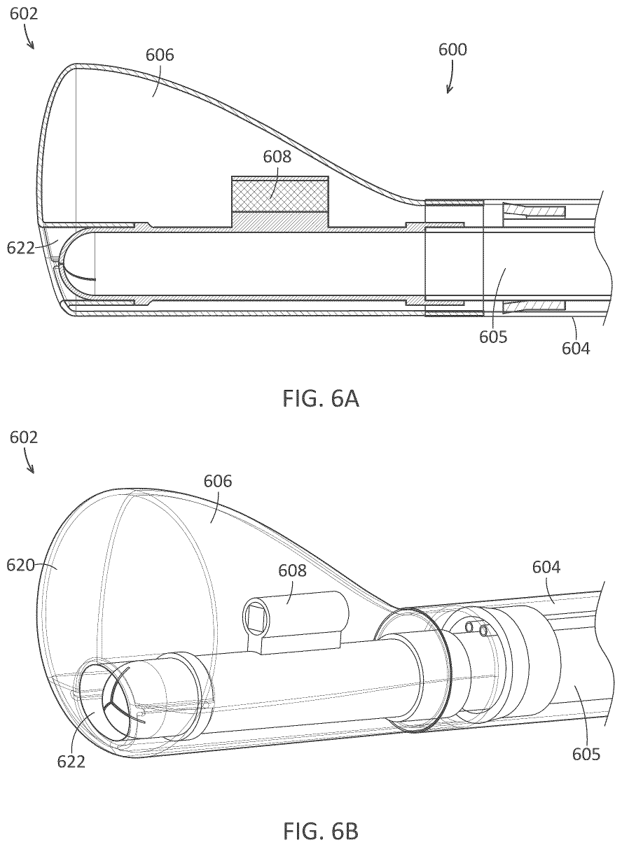

Moving to FIGS. 6A and 6B, a cross section and perspective view of a distal end of an embodiment of an access device 600 comprising a BVM 602 are shown. The device 600 comprises a shaft 604 with a balloon 606 coupled to a distal end of the shaft 604. A working channel 605 extends through the shaft 604. A camera module 608 is positioned within the balloon 606. A flexible channel extension 616 extends from a distal end of the shaft.

In some embodiments, the flexible channel extension 616 comprises silicone, in some embodiments.

The balloon comprises an asymmetric bulb shape with the bulk of its volume positioned above the shaft 604 and working channel 605. The balloon can increase in diameter from its proximal end to its distal end. The balloon comprises a curved distal end providing the distal window for the camera module 608. The distal window 620 can be sized and shaped to approximate a desired field of view for the camera/lighting/imaging module 608.

A proximal end of the balloon can be generally tubular shaped with a generally circular cross section to correspond to a distal end of the sheath 604.

The balloon is configured with a channel 622 shaped to accommodate a distal end of the flexible channel extension 616. The balloon 606 can be coupled to the flexible channel extension 616 around the channel 622.

As described above, a first portion of a distal end of the balloon is shaped to provide alignment and access to a working channel 605 within the sheath 604 and a second portion of the distal end of the balloon is sized and shaped to correspond to a desired field of view for the camera/lighting/imaging module 608.

An inlet channel 610 and outlet channel 612 extend along the shaft 604 and are in fluid communication with the balloon 606.

In some embodiments, the camera module 608 is positioned on a top portion of the flexible channel extension 616.

In some embodiments, the balloon comprises silicone. Other materials (e.g., Pebax®, Nylon, Polyester/PET, special compounded blends such as TPU/Pebax® and Pebax®/nylon, multilayer structures, etc.) are also contemplated.

In some embodiments, a durometer of the material can be about shore 80-90 A (e.g., shore85 A). Other durometers (e.g., shore 20-60 A, 30-40 A, 35-45 A, 45-75 A, 75-85 A, 60-100 A, 70-90 A, etc.) are also contemplated.

The balloon can comprise a thickness of about 0.20-0.25 mm (or about 0.15-0.30 mm, or about 0.10-0.35 mm, etc.).

In some embodiments, the balloon comprises a uniform thickness. In some embodiments, the thickness of the balloon varies.

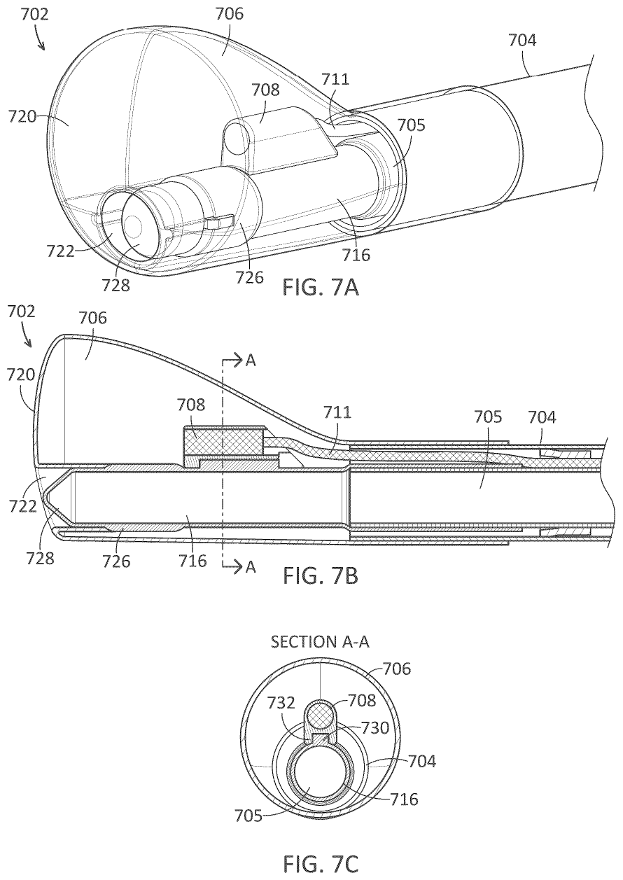

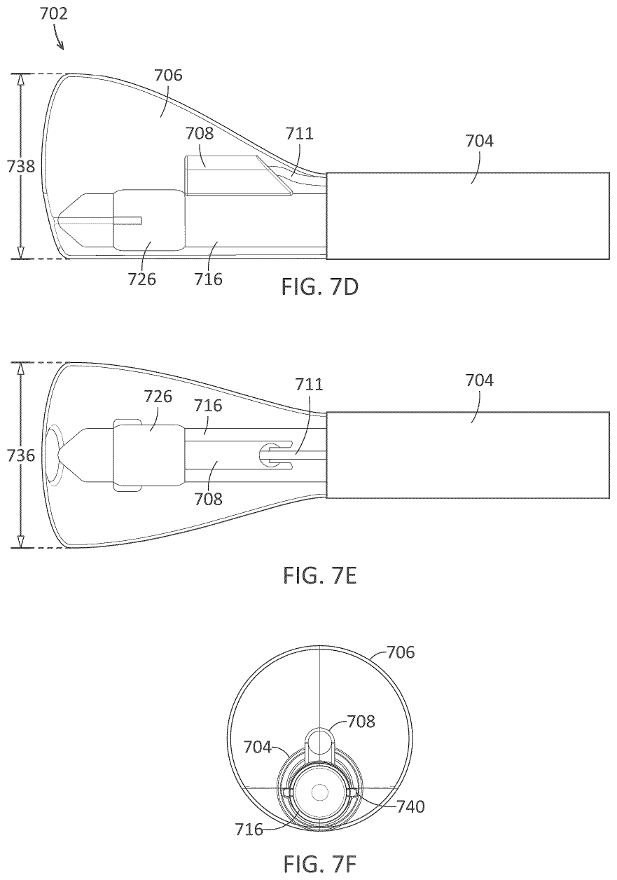

Referring now to FIGS. 7A-7F, various views of another embodiment of an access device 700 with a BVM 702 are shown. FIGS. 7A and 7B show isometric and side cross-section views, respectively, of another embodiment of a device 700 comprising a BVM 702. Unless otherwise described the device 700 comprises features similar to the device 600.

The device 700 comprises a shaft 704 through which a working channel 705 extends. A flexible extension 716 is positioned at a distal end of the shaft. The flexible extension 716 can be coupled to the shaft.

In some embodiments, the flexible extension 716 is transparent. Non-transparent (e.g., stainless steel or nitinol configurations are also contemplated.

The distal end of the flexible extension 716 can comprise a distal tip 728 that tapers to a rounded distal point. Other shapes are also possible (e.g., as shown in FIGS. 6A and 6B). The distal tip.

The camera module 708 is positioned on the flexible extension 716.

The balloon comprises an asymmetric bulb shape with the bulk of its volume positioned above the flexible extension 716. The balloon 706 can increase in diameter from its proximal end to its distal end. The balloon comprises a rounded distal end with a curved profile providing the distal window for the camera module 608. The distal window 720 can be sized and shaped to approximate a desired field of view for the camera/lighting/imaging module 708.

A proximal end of the balloon can be generally tubular shaped with a generally circular cross section to correspond to a distal end of the sheath 704.

The balloon is configured with a channel 722 shaped to accommodate a distal end of the flexible channel extension 716. The balloon 706 can be coupled to the flexible channel extension 716 around the channel 722.

As described above, a first portion of a distal end of the balloon is shaped to provide alignment and access to a working channel 705 within the sheath 704 and a second portion of the distal end of the balloon is sized and shaped to correspond to a desired field of view for the camera/lighting/imaging module 708.

An inlet channel and outlet channel (not shown) can extend along the shaft 704 and be in fluid communication with the balloon 706.

FIG. 7C shows a section view of the device through line A-A shown in FIG. 7A. The shaft 704 and the balloon 706 are visible in this view. The camera module 708 is shown positioned on the flexible extension 716. As shown in FIG. 7C, the flexible extension can have a feature 734 (e.g., a ridge, projection, etc.) configured to mate with a corresponding feature 732 (e.g., groove, depression, etc.). Corresponding mating features can help to strengthen the bond between the two components. The camera module 708 and the flexible extension 716 can be bonded together. Other configurations are also possible (e.g., glued together). A band 726 may also be used to join the components together.

FIGS. 7D-F show side, top and end views, respectively, of the access device 700 with BVM 702. These views show the device shaft 704 and flexible extension 716. The camera module 708 is positioned on the flexible extension. The camera module 708 connector 711 extends proximally from the camera module through the shaft 704.

The top view of FIG. 7E shows that the balloon 706 is generally symmetrical and coaxial with the sheath 704 along its width 736. The side view of FIG. 7D shows that the balloon 706 is not symmetric or coaxial with the shaft 704 along its height 738. Instead, more of the balloon is positioned above the shaft 704 and working channel 705.

The flexible extension of FIGS. 7D-F comprises a protrusion/ridge 740.

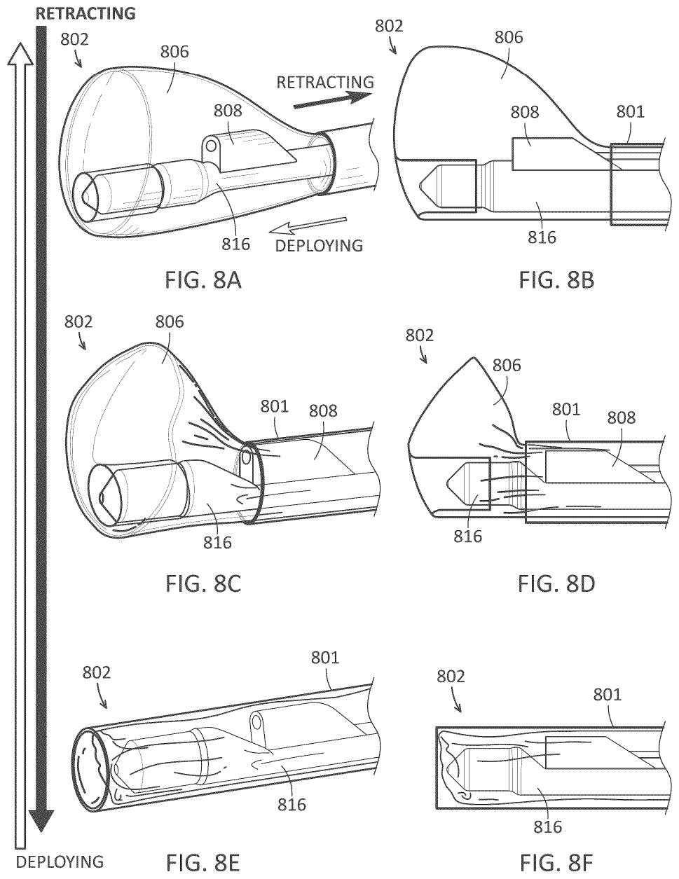

Moving now to FIGS. 8A-8E, deployment of an embodiment of a BVM similar to that shown in FIGS. 6A-7B is shown. FIGS. 8A and 8B show isometric and side views, respectively, of a fully deployed BVM 802. The BVM 802 can be delivered through an access sheath. In the fully deployed configuration, the camera 808 is in the desired position. The camera module 808 can be held by the balloon 806 of the working channel extension 816.

FIGS. 8C and 8D show isometric and side views, respectively, of a partially deploying or recovering BVM 802. A recovering BVM refers to BVM as it is being pulled back into an access sheath. FIGS. 8C and 8D show the working channel 805 being advanced out of the access sheath 804. The retraction of the working channel 805 causes pulls the balloon 906 and camera module 808 along with the working channel 805 as the balloon 806 and, in some embodiments, the camera module 808, is coupled to the working channel 805. For the same reason, advancement of the working channel 805 out of the sheath 804 causes the balloon 806 and camera module 808 to also be advanced out of the sheath 804. Once the BVM 802 is advanced out of the sheath 804, the balloon can be inflated, orienting the camera in the desired position.

Moving to FIGS. 8E and 8F, isometric and side views of a stowed or retracted state of a BVM 802 are shown. In the stowed and/or retracted configuration, each part of the BVM and the working channel extension 805 are housed within the sheath 804 for device insertion or removal.

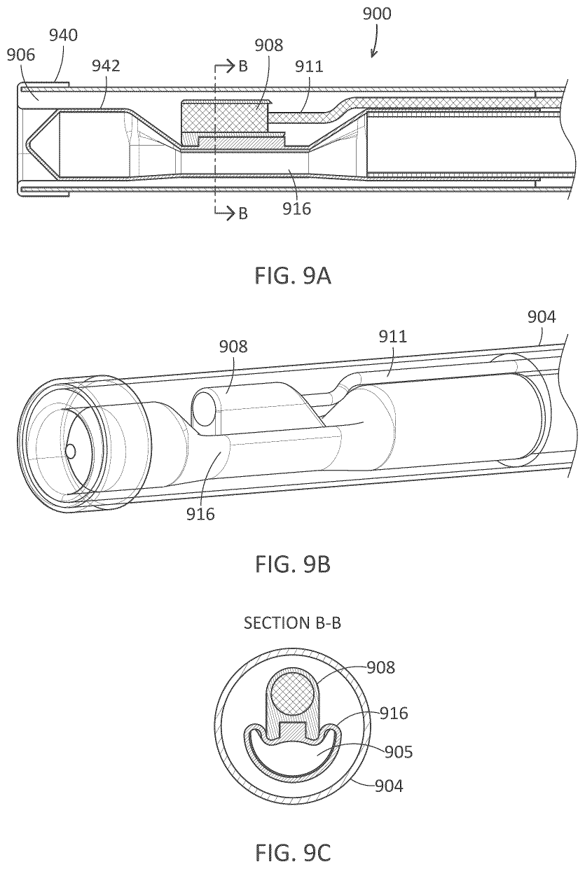

Referring now to FIGS. 9A-9C, side cross section, isometric, and a section views, respectively of a distal end of an access device 900 comprising a BVM 902 in a stowed configuration are shown. The flexible extension 916 is shown positioned within the sheath 904. The portion 940 of the balloon 906 that becomes the proximal portion of the deployed balloon is shown extending from and folding over the sheath 904. The balloon 906 is in an inverted position relative to its deployed position within the sheath 904. The balloon 906 is not coupled to an interior portion of the sheath 906. The balloon 906 is coupled to a portion 942 of the flexible extension 916 that is positioned proximal to the distal tip 944 of the flexible extension. The flexibility of the extension 916 allows the portion of the extension around the camera module 908 to collapse, to accommodate the camera module 908 within the dimensions of the sheath 904. FIG. 9C shows a section view taken along line B-B of FIG. 9A. In this view, the extension 916 is shown as collapsed along a top section, resulting in a deformed and reduced working channel 906. This deformation of the extension 916 results in the camera module being able to be contained within the sheath for insertion and removal. The flexibility of the extension allows it to resume its more tubular shape once advanced out of the sheath, raising the camera module to a desired position.

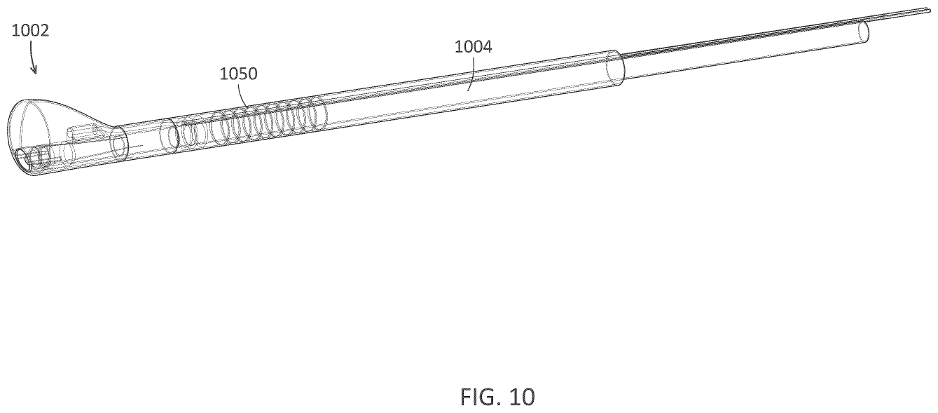

Moving now to FIG. 10, an isometric view of an embodiment of an access sheath 1000 comprising a BVM 1002 is shown. The access device comprises a region 1050 of controlled flexibility in the sheath.

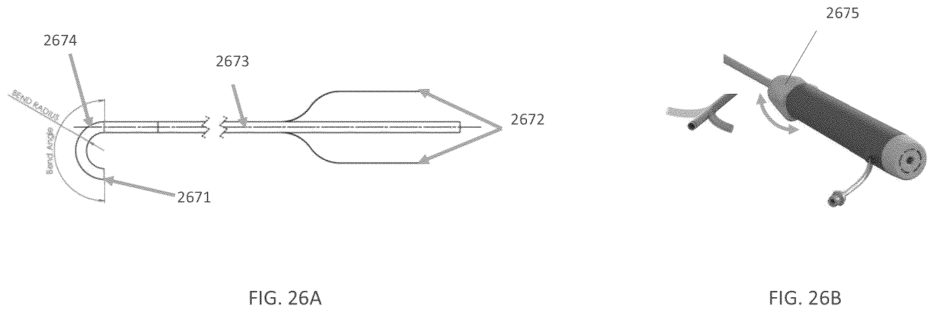

FIGS. 26A and 26B show an embodiment of a steering mechanism 2668 and control 2670 that can be used in the sheath. The sheath can comprise pull wires 2672 that can be anchored at the distal tip 2671. The sheath comprises a harder proximal section 2673 and a softer, steerable distal section 2674. A steering knob 2675 on the handle can be used to pull either of the pull wires 2672, resulting in articulation of the distal section 2674.

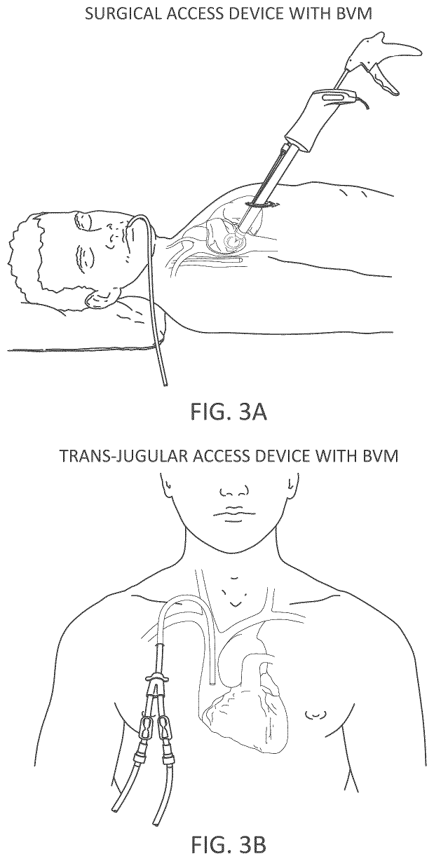

As described herein, the various alternative embodiments of the inventive surgical access device with a balloon based visualization module (BVM) may be used to address a wide range of clinical issues in a number of different medical procedures. FIG. 3A shows an embodiment of a surgical access device with a BVM being used in a procedure involving surgical access. FIG. 3B shows an embodiment of a surgical access device with a BVM being used in a procedure involving catheter based (e.g., trans-jugular) access. Accordingly, it is to be appreciated that in various embodiments the device shaft, working channel and balloon visualization module are adapted and configured for use in performing one or more steps of a minimally invasive, single port access, robotically assisted, interventional or surgical procedure to address the shortcomings of or clinical issues related to a variety of medical procedures. In some specific embodiments, the medical procedure may relate to minimally invasive mitral valve surgery, minimally invasive aortic valve surgery minimally invasive coronary artery surgery, surgical tachyarrhythmia treatment and left atrial appendage occlusion.

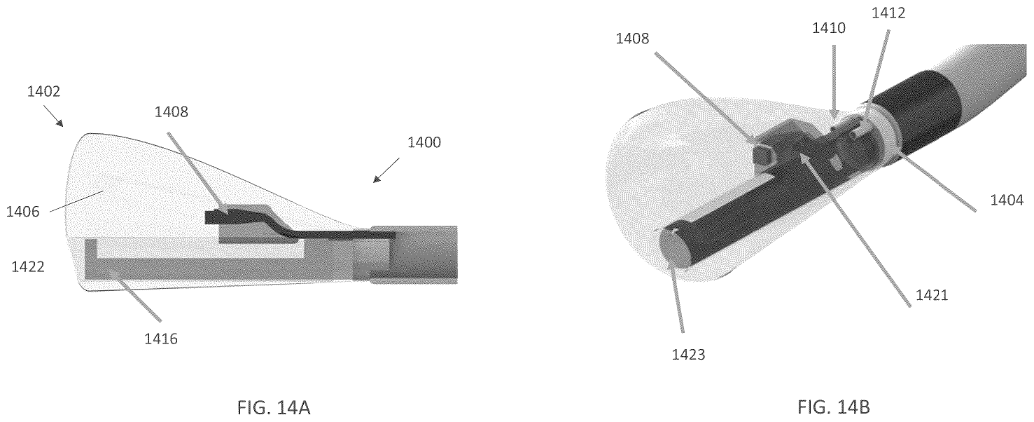

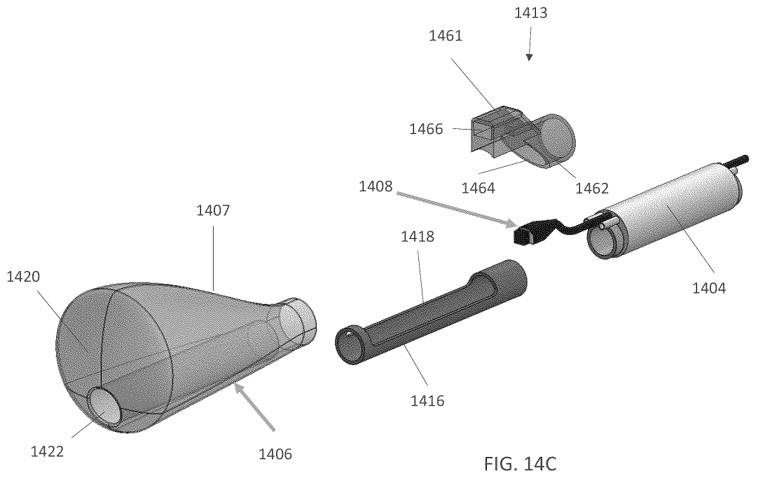

Referring now to FIGS. 14A and 14B, a side cross-sectional view and a perspective view of a distal end of another embodiment of an access device 1400 with a BVM 1402 is shown. FIG. 14C shows an exploded view of the distal end of the access device 1400. The device 1400 comprises a shaft 1404 with a balloon 1406 coupled to a distal end of the sheath 1404. A working channel 1405 extends through the sheath 1404. A camera module 1408 is positioned within the balloon 1406. A channel extension 1416 extends from a distal end of the working channel 1405.

The shaft 1404 can comprise a multilumen shaft. The lumens can extend within walls of the sheath or within a central lumen of the shaft. In some embodiments, some of the lumens are within the walls of the shaft and some of the lumens are within a central lumen of the sheath.

In some embodiments, the shaft is flexible.

The shaft can be 14F, in some embodiments. Other diameters (e.g., 10-18F, etc.) are also possible.

As shown best in FIG. 14C, fluid channels 1410, 1412 extend along the shaft 1404 and are in fluid communication with the balloon 1406. The fluid channels can be used for inflating, deflating and purging of the balloon 1406.

In some embodiments, the channel extension 1416 comprises an optically transparent material. In some embodiments, the channel extension 1416 comprises polycarbonate or other thermoplastics.

The channel extension 1416 can be transparent, in some embodiments.

As best shown in FIG. 14C, the channel extension 1416 comprises a cutout section 1418 extending along at least a portion of the channel extension 1416. The cutout section 1418 allows a space through which the camera module 1408 and a portion of the stowed balloon 1406 may be positioned and stored during delivery and retraction of the device 1400.

As shown in FIG. 14C, the cutout section 1418 may comprise a top portion (e.g., upper half, upper third, upper quarter, etc.) of the channel extension. Other configurations are also contemplated.

In some embodiments, the cutout section 1418 extends along about 60-90% of the length of the cutout section 1418.

In some embodiments, the cutout section 1418 begins at a location distal to the distal end of the shaft 1404.

In some embodiments, the cutout section 1418 ends at a location proximal to a distal end of the channel extension 1416.

The balloon comprises an asymmetric bulb shape with the bulk of its volume positioned above the shaft 1404 and working channel 1405. The balloon 1406 can increase in diameter from its proximal from a location at or near its proximal end to a distal end of the balloon. Along an upper surface or profile 1407 of the balloon, the balloon 1406 can increase in diameter at a greater rate than a bottom surface of the balloon. The balloon comprises a curved distal end providing the distal window for the camera module 1408. The distal window 1420 can be sized and shaped to approximate a desired field of view for the camera/lighting/imaging module 1408.

In some embodiments, the balloon material comprises a matching refraction index with the fluid (e.g., saline) used to fill the balloon to minimize unwanted reflections and distortions on the camera image.

A proximal end of the balloon can be generally tubular shaped with a generally circular cross section to correspond to a distal end of the shaft 1404. In some embodiments, a length of the generally tubular shaped proximal end is about 5 mm (or about 4-6 mm, 3-7 mm, 4.5-5.5 mm, etc.).

In some embodiments, a diameter of the generally tubular shaped proximal end is about 3.6-4.6 mm (or about 3.8-4.4 mm, 4.1 mm, 3.5-4.7 mm, 3-5 mm, 2.5-5.5 mm, etc.).

The balloon is configured with a channel 1422 shaped to accommodate a distal end of the channel extension 1416. The balloon 1406 can be coupled to the channel extension 1416 around the channel 1422.

As described above, a first portion of a distal end of the balloon is shaped to provide alignment and access to a working channel 1405 within the shaft 1404 and a second portion of the distal end of the balloon is sized and shaped to correspond to a desired field of view for the camera/lighting/imaging module 608.

In some embodiments, the camera module 1408 is positioned on a top portion of the balloon channel 1422. A connector 1411 extends from the camera module proximally. The connector 1411 can be configured to provide a data and/or electronic connection.

In some embodiments, the device 1400 comprises a position tracking sensor 1421 (e.g., electromagnetic position tracking sensor) next to the camera module 1408.

As best shown in FIG. 14C, a camera mount 1413 can be used to mount the camera module 1408 to the balloon 1406. The camera mount 1413 comprises an upper portion 1461 and a lower portion 1462. The lower portion 1462 can comprise a lumen or aperture 1464 configured to be positioned over the balloon channel 1422 within the balloon. The upper portion 1461 comprises an aperture, slot, or lumen 1466 configured to receive the camera module 1408. In some embodiments, the upper portion 1461 can be coupled to a distal portion of the lower portion 1462, at its distal end. The lower surface of the upper portion 1461 can comprise a shape configured to conform to the upper surface of the balloon channel 1422.

In some embodiments, the balloon comprises polyurethane (e.g., pellethane 80AE thermoplastic polyurethane). Other materials (e.g., silicone, Pebax®, Nylon, Polyester/PET, special compounded blends such as TPU/Pebax® and Pebax®/nylon, multilayer structures, etc.) are also contemplated.

In some embodiments, a durometer of the material can be about shore 80-90 A (e.g., shore85 A). Other durometers (e.g., shore 20-60 A, 30-40 A, 35-45 A, 45-75 A, 75-85 A, 60-100 A, 70-90 A, etc.) are also contemplated.

The balloon can comprise a thickness of less than about 0.1 mm (or about 0.05-0.01 mm, or 0.05-1.5 mm, etc.).

In some embodiments, the balloon comprises a uniform thickness. In some embodiments, the thickness of the balloon varies. For example, the balloon channel 1422 can comprise a greater thickness (e.g., 0.1 mm, 0.2 mm, 0.15 mm, etc.), in some embodiments.

In some embodiments, the balloon comprises a non-compliant material. In such embodiments, the balloon material does not stretch when pressurized.

In some embodiments, the balloon comprises an optically clear material. Non-optically clear materials are also contemplated.

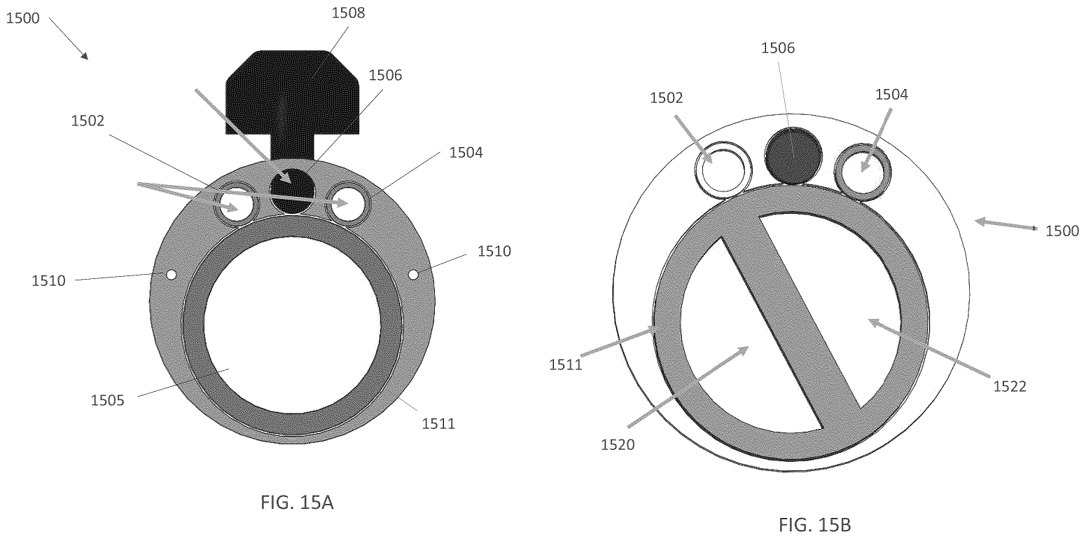

Moving now to FIG. 15A, a section view of an embodiment of a multilumen shaft 1500 (e.g., similar to multilumen shaft 1406) is shown. The shaft can comprise a flexible material. In some embodiments, the shaft is laminated. An outer portion of the shaft can comprise a jacket. The jacket can comprise a thermoplastic tube.

The shaft comprises five lumens 1502, 1504, 1506, 1508 embedded within its walls. Lumens 1502, 1504 can be configured to receive fluid lines (e.g., fluid lines 1410, 1412). In some embodiments, the fluid lines comprise plastic (e.g., a plastic with a high melting point).

Lumen 1506 can be configured to receive a connector (e.g., connector 1411) for the camera module. Lumens 1510 can, in some embodiments, be configured to receive position tracking sensor wiring. While the lumens are shown embedded within the shaft wall, it will be appreciated that, in some embodiments, they can extend along an interior of the shaft 1406.

In some embodiments, the lumens 1502, 1504, 1506 can comprise an inner diameter of about 0.4-0.6 mm (or about 0.3-0.7 mm, about 0.45-0.55 mm, about 0.5 mm, etc.).

In some embodiments, the working channel 1505 extending through the shaft comprises a diameter of about 8-9F (or about 7-10F, 6-11F, etc.).

In some embodiments, an outer diameter of the shaft is about 15F (or about 14-16F, about 13-17F, etc.).

A tube 1511 can extend through the central lumen, forming the working channel 1505. In some embodiments, the tube 1511 comprises a thermoplastic. Other materials are also contemplated (e.g., a metal). The tube 1511 can comprise a braid configuration

In some embodiments, additional lumens (e.g., for sensors, light emitters, heat transfer, etc.) are included.

It will be appreciated that lumens or tubes described herein an comprise round, ovular, flat or any other shape through which fluid and other items can travel

It will be appreciated that more than one working channel (e.g., 2, 3, 4, 5, 6, etc.) is contemplated, in some embodiments. FIG. 15B shows a section view of a shaft 1500 comprising a dual lumen working channel tube 1511 having two lumens 1520, 1522. The shaft 1500 comprises a wall 1524 separating the two lumens 1520, 1522. Fluid channels 1502, 1504 and a lumen 1506 for receiving a camera module connector are also shown.

The multilumen shaft can be constructed by inserting the lumens or tubes for the working fluid channels and camera module connector on an external portion of the working channel tube. The working channel tube can be positioned on a mandrel. The jacket tube can be positioned over all of the tubes/lumens. Heat shrink can be applied over the assembly. Upon application of heat, the jacket tube melts around the internal tubes and lumens. After heat shrink removal, the individual components form a unified multilumen shaft.

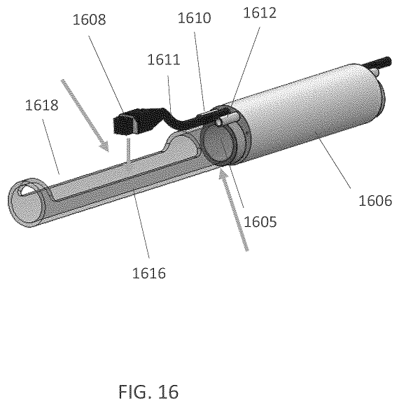

Referring now to FIG. 16, a perspective view of a distal end of an embodiment of an extension channel 1616 (e.g., like the extension channel 1416) and shaft 1606 (e.g., like the shaft 1406) is shown. The shaft 1606 and working channel 1605 is visible in FIG. 16. Fluid channels 1610, 1612 and camera module connector 1611 extend through the shaft 1606. The camera module is shown connected to the connector 1611. The extension 1616 extends from and is coupled to a distal end of the channel 1605. In some embodiments the extension 1616 extends from a distal end of the shaft.

In some embodiments, the working channel 1605 extends past a distal end of the shaft before being coupled to the extension 1616.

The channel extension 1616 can advantageously provide axial strength to the balloon so that it can be pushed against the target tissue without the balloon collapsing.

In some embodiments, the camera module 1608 blocks a portion of the working channel 1605 and/or channel extension 1616 when in the stowed configuration. As such, the working channel is not fully available or until the balloon is sufficiently inflated to lift the camera module out of the working channel path. In some embodiments, the working channel is sufficiently open to allow passage of some tools (e.g., a guidewire, dilator) when the BVM is in the stowed configuration.

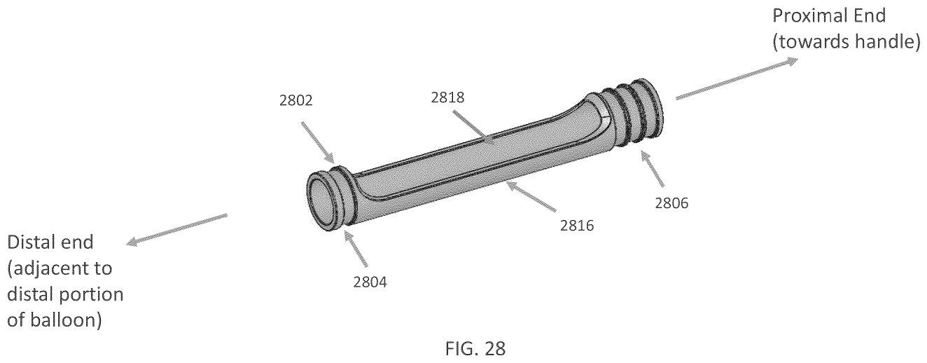

Referring now to FIG. 28, an embodiment of a channel extension 2816 is shown. The channel extension can comprise a distal mating feature 2802, a ring 2804, a cut out portion 2818, and a proximal mating feature 2806.

The distal mating feature 2802 and proximal mating feature 2806 can be used to aid in the coupling of the channel extension with other portions of the device. Joining the channel extension using the mating feature can be performed using, for example, bonding, welding, or other joining methods. The mating feature can comprise grooves, threads, snap fits, etc.

In some embodiments, the channel extension comprises a transparent material. Non-transparent materials are also contemplated.

The channel extension can be rigid or flexible.

In some embodiments, the channel extension comprises a thermoplastic. Other materials are also contemplated (e.g., metal or ceramic).

The channel extension can comprise a diameter of about 8F or 2.6 mm (or about 2.1-3.1 mm, 2-3 mm, 1.5-3 mm, etc.).

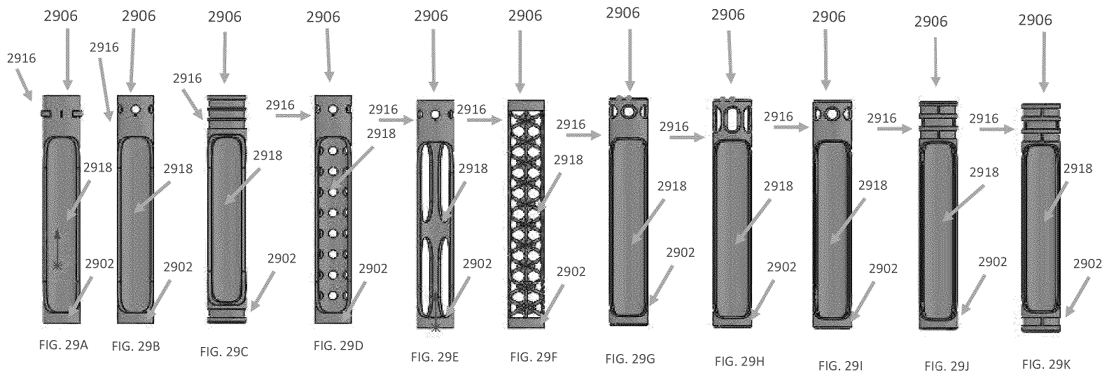

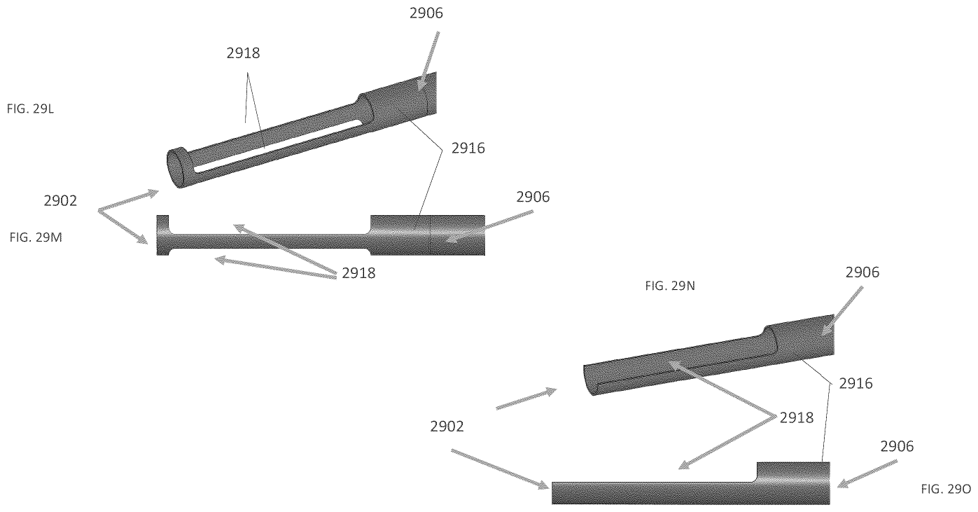

FIGS. 29A-29K show various embodiments of channel extensions 2916 comprising various proximal 2906 and distal 2902 mating features. The mating features 2906, 2902 can comprise tabs or protrusions (FIG. 29A, B, D, E, G, H, I), ridges (FIG. 29C, 29J, 29K), apertures (FIG. 29B, 29D, 29E, 29G, 29H, 29I) or any combinations thereof. The ridges can be full round as shown in FIG. 29C or segmented as shown in FIGS. 29J and 29K. The cut-out feature 2918 can extend along the body of the channel extension between the mating features 2906, 2902. The embodiments of FIGS. 29A-29K illustrate a top down view of an elongated generally rounded rectangular shaped upper portion of the cut-out feature 2918. FIG. 29F illustrates an embodiment where the proximal and distal mating features have been minimized to maximize the size of the cut-out feature 2918. The lower portion of the cut-out portion 2918 may be solid as shown in FIGS. 29A, 29B, 29C, 29G, 29H, 29I, 29J and 29K. Additionally or optionally, the lower portion of the cut-out portion 2918 may include various patterns (repeating ovular cutouts, aperture grid, lattice structure, etc.) as shown in FIGS. 29D-29F. The cut-out portion 2918 shape, size and dimensions are related to the size of the balloon and balloon visualization module when in a stowed configuration. A larger balloon may lead to channel extensions with a greater length between the proximal and distal mating features. A larger balloon visualization module or multiple mode or multiple camera balloon visualization module may lead to greater modification and variation of the length and shape or contours of the cut-out portion 2918. Still other channel extension embodiments may modify the channel extension cut-out portion bottom section for variations in structural characteristics such as to either modify flexibility or rigidity when in use with the balloon. Additionally or optionally, modifications to the cut-out portion bottom portion may be provided to enhance the visual field (i.e., having more open space in the bottom portion). In other configurations the channel extension is formed from optically transparent material selected to minimize interference with the visual field of the camera or optics of the balloon visualization module.

FIGS. 29L and 29M show perspective and side views of an embodiment of a channel extension 2916. The channel extension 2916 has a proximal mating feature 2906 and a distal mating feature 2902. The cutout portion 2918 has an upper and a lower portion that produces side rails extending from the proximal end to the distal end terminating in the distal mating features 2902. The use of a cut out portion like 2918 provides the most space for storage of the balloon and balloon visualization module as well as maximizing the visual field of view by removing the lower portion of the channel extension.

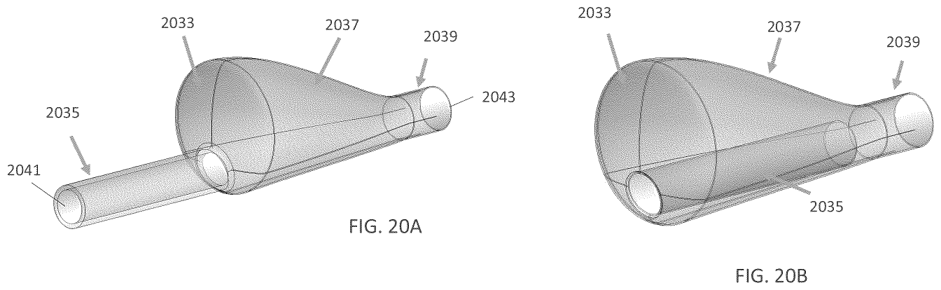

FIGS. 29N and 290 show perspective and side views of a channel extension 2916. The channel extension 2916 includes a proximal mating feature 2906. The channel extension 2916 includes a single cutout portion 2918 forming a half-pipe configuration. There is no distal ring for the distal mating feature as in FIG. 29L. Instead the balloon distal leg could be attached to the distal portion of the bottom surface of the channel extension (see e.g., FIGS. 20A-24D).

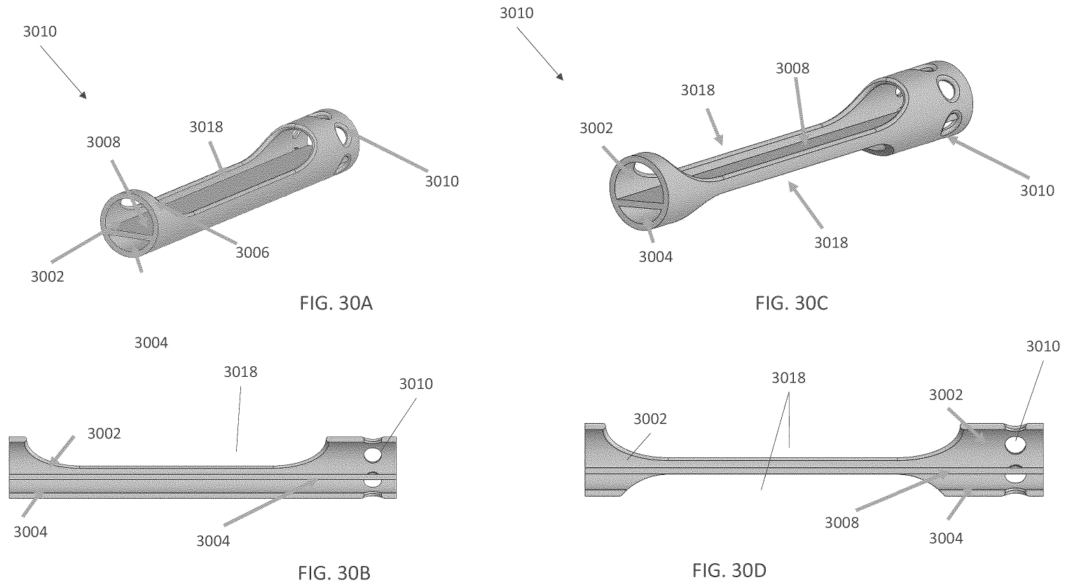

Referring to FIGS. 30A-30D, various views of embodiments of channel extensions 3016 comprising multiple lumens are provided.

FIGS. 30A and 30B show perspective and side section views, respectively, of a channel extension 3016 comprising two lumens 3002, 3004. The channel extension 3016 comprises a single cutout 3018 positioned within the top lumen 3002. The channel extension 3016 comprises a distal ring 3006. A wall 3008 separates the two lumens 3002, 3004. The channel extension 3016 comprises a proximal mating feature 3010 comprising a ring of apertures.

FIGS. 30C and 30D show perspective and side section views, respectively, of a channel extension 3016 comprising two lumens 3002, 3004 and also two cutout portions 3018, with one positioned in each lumen. The channel extension 3016 comprises a distal ring 3006. A wall 3008 separates the two lumens 3002, 3004. The channel extension 3016 comprises a proximal mating feature 3010 comprising a ring of apertures. It will be appreciated that more than two lumens (e.g., 3, 4, 5, 6, etc.) are also contemplated).

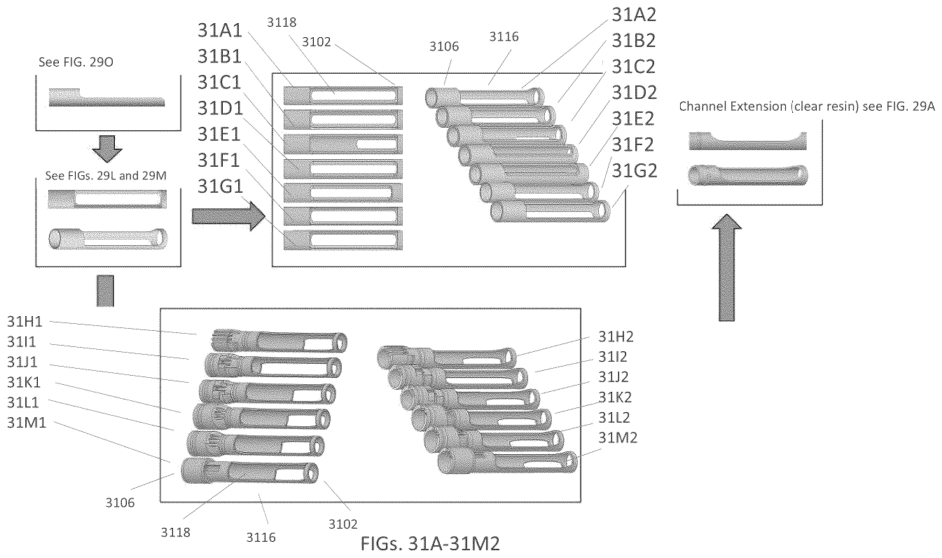

FIG. 31 shows various embodiments and configurations for a number of different possible channel extension cutout portions and proximal and distal mating feature configurations. In one general aspect, a channel extension may be considered as having a generally cylindrical overall shape. Depending on the specific embodiment, a proximal end portion of a channel extension is used to couple to a distal end portion of the elongate body. Additionally, or optionally, the proximal end portion is used to provide physical connection points for balloon inflation lines, balloon deflation lines, camera power and imaging connections as well as other control lines as needed depending upon the specific capabilities of the balloon and balloon visualization module. The proximal end portion may also be adapted and configured to be coupled to the proximal end of the balloon (See for example FIGS. 14A, 14B, 14C, or 33). In still other aspects, the proximal end is adapted and configured to couple to the distal end of the distal end of the balloon. In this aspect, it is the everted end of the balloon distal end that is coupled, bonded or otherwise joined to the proximal end of the channel extension. As such, the proximal end and the mating feature 3106 is provided for these and other purposes and may have a number of different configurations. The distal end of the channel extension is also used for coupling to the balloon distal end as well as for providing additional structural and longitudinal support to the balloon. The upper portion of the cut-out 3118 is used to provide space for storage of the balloon and balloon visualization module. The lower portion of the cut-out 3118 is used to adjust the structural characteristics or to also remove material that may otherwise block or impede the field of view of the camera in the balloon visualization module. The various embodiments shown in FIG. 31 include an elongated upper cut out portion that results in a proximal end portion and a distal ring portion. The lower cut out portion is then either a full length as in FIGS. 31A1/2, 31B1/2, 31E1/2, 31F1/2, 31G1/2 or a partial length as in FIG. 31D1/2. The resulting channel extensions have generally cylindrical ends with the distal end being positioned at the end of a pair of side rails of various sizes based on the relative dimensions of the upper and lower cutouts. The extremes of this aspect may be appreciated by reference to a comparison of the width of the side rails in FIG. 31G2 to those of FIG. 31C2.

Additional aspects of a various channel extension embodiments may be appreciated with regard to the front perspective views FIGS. 31H1-31M1 and rear perspective views of FIGS. 31H2-31M2, respectively. Each of the channel extension embodiments illustrated has a longated upper cut-out as in the embodiments of FIGS. 31A1-31G2. The distal end mating feature is also similarly arranged as a ring and side rails or walls of various sizes are also seen. The lower portion cut out may be the full length as in FIG. 3111 or only in the distal portion or distal half of the lower portion as best seen in FIGS. 31H1, J1, K1, L1 and M1. Additionally, the proximal end portions of FIGS. 31H-31M2 include functional features not present in the embodiments of FIGS. 31A1-31G2. The proximal end portion may include enhancements for coupling to the various portions of the balloon or for providing mechanical connections to the balloon inflations, balloon deflation and camera, power or imaging connections.

Moving to FIGS. 17A and 17B, a side cross-sectional view of just a balloon 1606 and of the balloon visualization module 1602 including the balloon 1602 are shown. Unless described otherwise, the balloon and balloon visualization module can be similar to that described above (e.g., balloon 1406 and BVM 1402).

At its proximal end, 1701, the balloon comprises a tubular profile. From there, the upper surface 1407 extends radially outwardly from a longitudinal axis of the channel 1722. The upper surface 1407 can extend radially outwardly from the longitudinal axis 1723 of the channel 1722 by an angle about 40-50° (or about 45, about 30-60°, 25-65°, 20-70°, etc.

The lower surface or profile 1725 of the balloon can extend from the proximal portion at an angle of about 5-20° (or about 5-15°, 5-10°, 5-25°, 10-20°, 10-25°, 15-25°, etc.).

The distal portion of the balloon provides a contact visualization surface 1720 of the balloon 1706. The contact visualization surface 1720 can comprise a rounded profile.

FIG. 17B shows the field of view 1727 of the camera module 1708. As shown, in FIG. 17B, when the camera module is in the deployed position, the field of view of the camera module 1708 includes most of the contact visualization surface. In some embodiments, the field of view of the camera module includes at least 95% of the contact visualization surface (or about 90%, or about 85%, or about 80%, or about 75%, or about 70%, etc.)

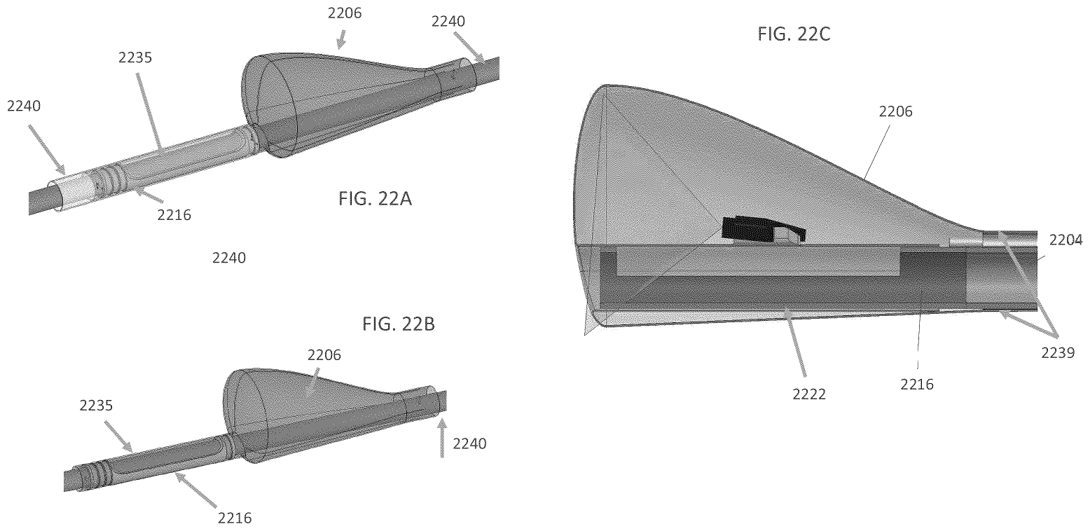

The balloon channel 1722 wraps around the channel extension 1716. The asymmetric constructions allows the balloon to wrap closely around the useful volume of the device to minimize blood displacement and flow disturbance (e.g., within the heart). This construction is described in further detail below (e.g., with respect to FIGS. 22A-22C.

In some embodiments, the balloon may be symmetrical (e.g., spherical, conical, cylindrical, etc.). Such balloons can be used for specialized procedures. They may also provide ease of manufacturing.

In some embodiments, the camera module may be provided at an angle other than normal to the working channel. In such embodiments, a contact visualization surface can be provided on the side of the balloon with the angled camera placement.

As shown in FIG. 17A, in some embodiments, the height 1702 of the balloon in an inflated configuration can be about 12-14 mm (or about 13 mm, or about 11-15 mm, or about 12.75-13.23 mm, or about 10-13 mm or about 11-13 mm, or about 11-14 mm or about 12-15 mm, etc.).

In some embodiments, a diameter of the balloon channel 1722 can be about 3-3.8 mm (or about 3.3-3.5 mm, or about 3.4 mm or about 3-4 mm, or about 2-5 mm, or about 2.5.4.5 mm, etc.).

In some embodiments, a length of the balloon channel 1722 is about 16-17.4 mm (or about 16.5-16.9 mm, or about 16.7 mm, or about 15-18.4 mm, or about 14-19.4 mm, or about 15.5-18.9 mm, etc.).

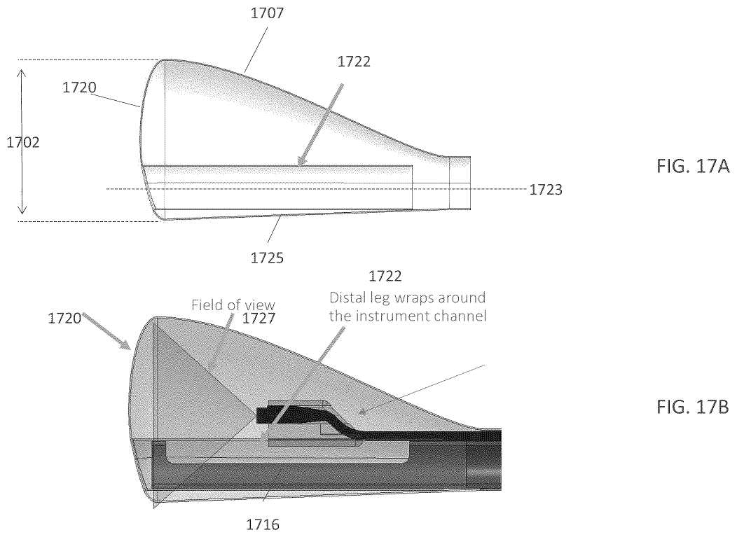

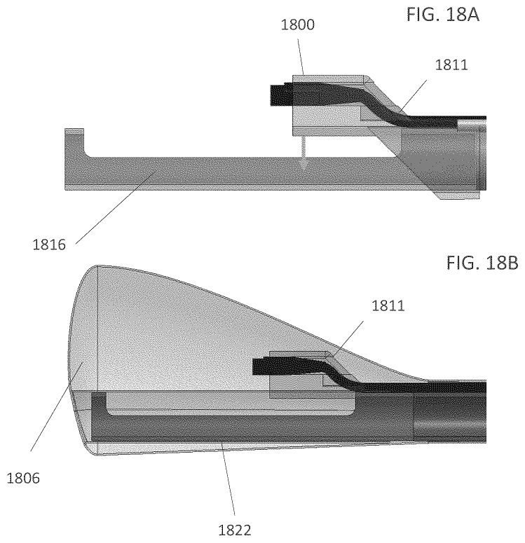

Referring now to FIGS. 18A and 18B, two configurations of a camera module mount 1811 are shown. The camera module mount 1811 is shown in use with a device such as device 1400. In FIG. 18A, the camera module mount 1811 is shown mounted to the channel extension 1816. In FIG. 18B, the camera module mount 1811 is shown mounted to the balloon channel 1822.

The camera module mount is configured to hold the camera in the right location for visualization while allowing the camera module to flex within the instrument channel during insertion and withdrawal.

The camera module mount can be configured to hold the camera horizontally or in an angle depending on the requirements of the procedure and balloon shape.

In some embodiments, the camera module comprises a 400×400 pixel color camera module. The camera module can comprise a field of view of about 110° in air and about 80° in water. Other fields of view (e.g., 100-180°, 100-170°, 100-160°, 100-150°, etc.) are also contemplated.

In some embodiments, the camera module comprises an integrated camera module and lighting (e.g., LED) system.

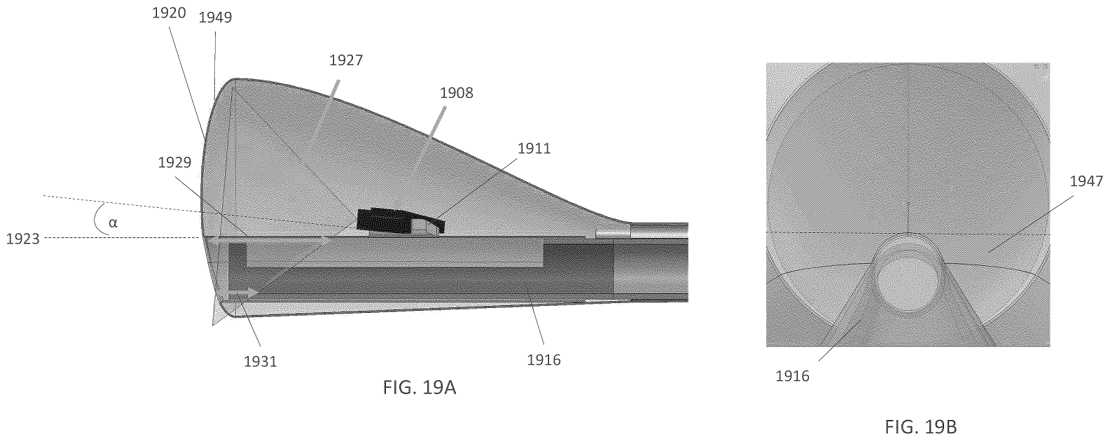

Referring now to FIGS. 19A and 19B, a side view of an embodiment of a BVM 1902 is shown. The camera module 1906 is positioned on a camera mount. As shown in FIG. 19, the camera module can be positioned such that it is at an angle relative to the longitudinal axis of the channel extension 1916. For example, the angle can be about 0-10° (or 1-20°, 5-10°, 5-15°, 5-20°, etc.).

In some embodiments, a distance of the camera module from the contact visualization surface 1920 and the angle α relative to the longitudinal axis 1923 of the channel extension 1916 can be chosen to allow the field of view 1927 of the camera module 1908 to correspond with and/or cover the perimeter of the contact visualization surface 1920.

The distance between the camera module and the contact visualization surface can be about 8-13 mm, in some embodiments.

The positioning of the camera module 1908 can allow a portion of a distal end of the channel extension 1916 to be visible by the camera module. In some embodiments, about 6-7 mm (or about 7 mm, about 5-9 mm, about 6.5-7.5 mm, etc.) of a top portion 1929 of the distal end of the channel extension is within the field of view 1927. In some embodiments, about 2-3 mm (or about 1-4 mm, 1.5-3.5 mm, 2.5 mm, etc.) of a bottom portion 1931 of the distal end of the channel extension 1916 is within the field of view 1927.

FIG. 19B shows a view from the camera module 1908. As shown in FIG. 19B, in some embodiments, the channel extension 1916 is visible at a bottom portion 1947 of the field of view. The channel extension 1916 can comprise about ⅓ (or about 30-35%, 30-40%, 25-35%, 20-40%, 15-45%, etc.) of a bottom portion 1947 of the field of view of the camera module.

The window 1920 shape and size can be optimized depending on the goal of the procedure. For example, for a procedure at the apex of the heart, a smaller field of view with a tighter distal radius 1949 may be used than when performing a procedure at the valves, during which a larger field of view with a bigger radius 1949 may be desired.

Referring now to FIGS. 20A and 20B, embodiments of a balloon 2002 during two steps of the manufacturing process are shown. FIG. 20A shows the balloon 2002 as manufactured. The shape of the balloon can be set by a blow mold tool. As manufactured, the balloon 2002 comprises an optical window 2033, a distal leg 2035, the balloon body 2037, and a proximal leg 2039. The distal leg 2035 comprises a generally tubular shape with an open distal end 2041. The proximal leg 2039 comprises a generally tubular shape with an open proximal end 2043. The proximal leg 2039 can be shorter than the distal leg.

FIG. 20B shows the balloon 2002 as assembled. The distal leg 2035 is folded backwards, such that it is now proximal to the optical window 2033.

The balloon can be in four states: a deflated state, an inflated state, an underinflated state, and an overinflated state. The different states of the balloon can be achieved by flowing fluid (air, saline, or other fluids) into the balloon using one or more fluid lines. Purging of the balloon can be performed by continuously flowing fluid through the balloon.

When the balloon is in the underinflated, inflated and overinflated state it displaces blood from the field of view of the camera system. The BVM is deployed through the access sheath into the target area after which the balloon is (under/over) inflated. In this state, when the Optical Window of the balloon is pushed to the surface tissue of the hearth Contact Optical Visualisation is realised. The balloon pressure can be set so that the distal window can gently comply onto the target tissue, reducing unwanted pressure on the target anatomy.

The inflation and purging of the balloon can be managed by a fluid pump or a syringe operator.

The distal leg 2035 of the balloon 2002 gets positioned around the channel extension and is ponded to a proximal end of the distal tip of the channel extension.

In embodiments in which the camera module is coupled to the balloon, it can be bonded (or otherwise attached) to the distal leg 2035 after it is folded back within the balloon.

The proximal leg 2039 of the balloon can be bonded (or otherwise attached) to an outer portion (e.g., outer jacket) of the shaft. The distal leg of the balloon is bonded (or otherwise attached) to a distal end of the working channel of the device at or near the opening 2041.

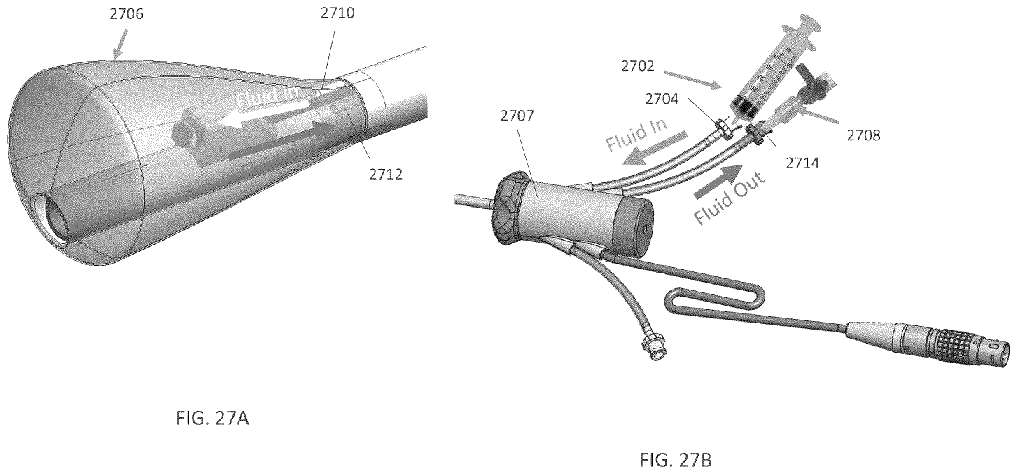

Referring now to FIGS. 27A and 27B, embodiments of methods for balloon inflation and deflation are shown. Prior to balloon inflation, the fluid channels 2710, 2712 and the balloon 2706 are purged to eliminate air trapped inside the channels and balloon. The balloon takes its full shape when it is in an inflated state. Its rigidity can be maintained by keeping constant pressure inside the fluid channels and the balloon body.

To purge, a syringe or fluid pump 2702 is connected to the fluid inlet 2704 at the handle 2707. A stopcock 2708 can be connected to the fluid outlet 2714 at the handle 2707. When purging, the stopcock 2708 is in an open position and fluid is continuously flushed by the syringe 2702 through the inlet 2704 into the balloon 2706 and out from the system through the outlet 2714. During purging, the balloon 2706 is not in its inflated configuration as its pressure is not constant. In some embodiments, about 100 ml is flushed through the fluid channels and the balloon during purging. Purging can be performed 2 times prior to balloon inflation. Fewer or more instances are also contemplated. Once purging is complete, the stopcock 2708 is moved to the closed position.

After purging the balloon 2706, the syringe 2702 or pump filled with fluid (e.g., saline) is connected to the fluid inlet 2704. A pressure sensor can be connected to the fluid inlet 2704. The stopcock 2708 is in the closed state at the fluid outlet 2714. Fluid is pushed from the syringe 2702 or pump through the inlet 2704 into the balloon 2706. Pressure can be continuously measured. When the pressure sensor senses that pressures starts to increase, fluid injection can be stopped. The balloon is now in its inflated state.

The overinflated state can be achieved by pushing more fluid into the balloon after the balloon reaches the inflated state.

To deflate, the stopcock 2708 is put in the closed state. Fluid is sucked back into the syringe 2702 or pump until negative pressure is sensed by the pressure sensor.

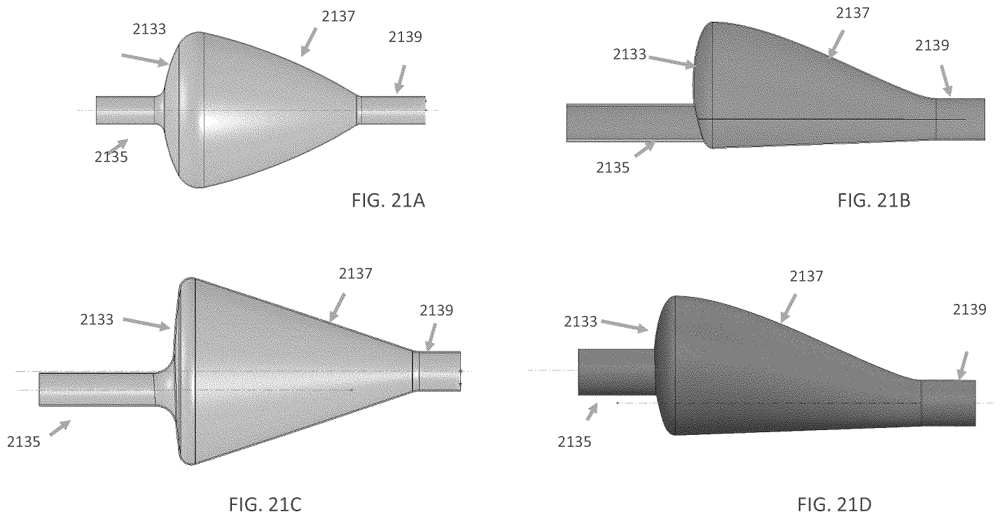

FIGS. 21A-21D show side views of various balloon embodiments that can be used with the devices described herein. As shown in these embodiments, the balloon may comprise a symmetric or asymmetric balloon body. The proximal and distal legs can be centered or not centered relative to one another. The proximal and distal legs can be in line or off axis relative to a longitudinal axis of the balloon. The optical window can be convex or concave. Any combination of these features is contemplated.

FIG. 21A shows an embodiment of a balloon comprising a symmetric balloon body 2137 with the balloon symmetrical along its longitudinal axis and the proximal and distal legs 2139, 2135 centered with one another. The optical window 3133 comprises a convex surface.

FIG. 21B shows an embodiment of a balloon with a balloon body 2137 that is asymmetric about its longitudinal axis (e.g., like balloon 2002). The proximal and distal legs 2135, 2139 are centered with one another. The optical window 2133 comprises a convex surface.

FIG. 21C shows an embodiment of a balloon with a balloon body symmetric about its longitudinal axis. The distal and proximal legs 2135, 2139 are not centered with one another. The proximal leg 2139 is centered with the longitudinal axis of the balloon. The distal leg is off axis with respect to the longitudinal axis of the balloon. The optical window 2133 comprises a concave surface.

FIG. 21D shows an embodiment of a balloon with a balloon body 2137 asymmetric about its longitudinal axis. The distal and proximal legs 2135, 2139 are excentered (i.e., not centered with one another). The distal leg 2135 is centered with the longitudinal axis of the balloon. The proximal leg 2139 is off axis with respect to the longitudinal axis of the balloon. The optical window 2133 comprises a convex surface.

FIGS. 22A-22C show embodiments of methods for connecting the balloon 2202 with the channel extension 2216. In FIG. 22A, the channel extension 2216 is shown positioned over a mandrel 2242. The balloon 2206 is shown with its distal leg 2235 positioned over the channel extension 2216. Heat shrink 2240 is shown positioned over the distal leg 2235. Upon application of heat, the balloon 2202 and channel extension bond.

In FIG. 22B, the distal leg 2235 of the balloon is positioned over the channel extension 2216. A mandrel 2242 is shown extending within the channel extension 2216. Glue is applied between the leg 2235 and the channel extension 2216.

Referring now to FIG. 22C, an assembled balloon with the channel extension 2216 coupled (e.g., laminated or bonded) to the distal leg, now the balloon channel 2222, is shown. The proximal leg 2239 of the balloon is laminated or bonded onto the outer diameter of the shaft 2204.

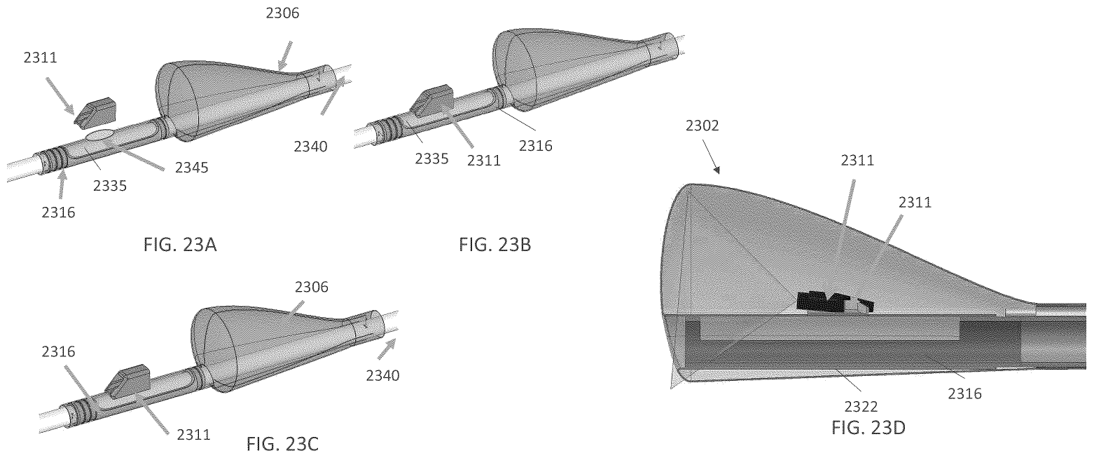

Moving now to FIGS. 23A-23C, embodiments of methods for connecting the camera support 2311 to the distal leg 2335 of the balloon 2302 are shown. These methods can be performed with or without a mandrel. FIG. 23A shows the channel extension 2316 positioned over a mandrel 2340. The distal leg 2335 is positioned over the channel extension 2316. Glue 2345 can be applied between the camera support 2311 and the distal leg 2335, coupling them, as shown in FIG. 23B.

FIG. 23C shows the camera support 2311 positioned over the distal leg 2335 and the channel extension 2316. Upon application of heat, the camera support 2311 (which can, for example, comprise plastic) laminates with the distal leg 2335 of the balloon 2306.

FIG. 23D shows an assembled BVM 2302, with the distal leg of the balloon pulled proximally within the balloon, forming the balloon channel 2322 comprising the channel extension 2316. The camera mount 2311 is shown coupled to the balloon channel 2322. The camera module 2308 is positioned within the camera mount 2311.

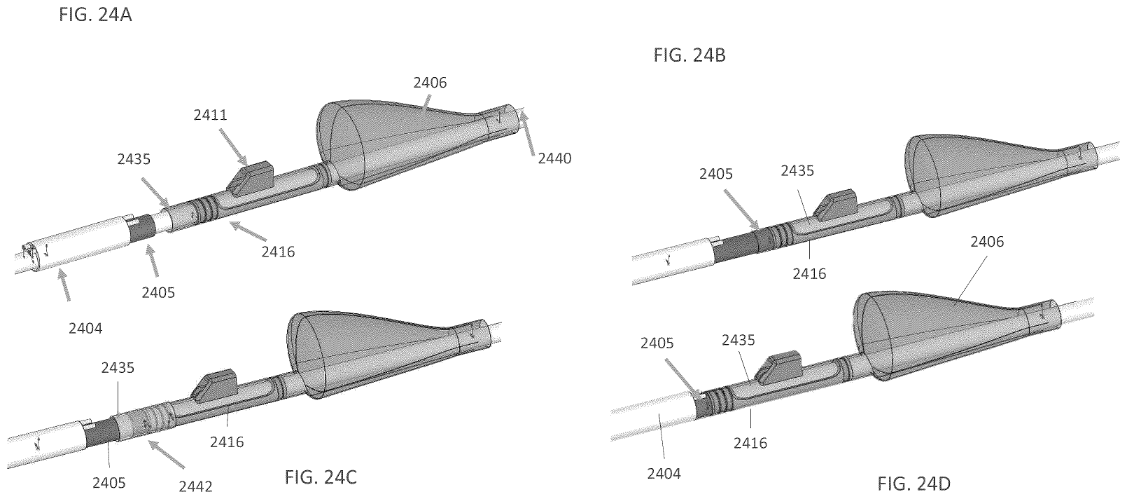

Referring now to FIGS. 24A-24D, embodiments of methods for connecting the shaft 2404 to the balloon 2406 and channel extension 2416 are shown.

FIG. 24A shows the balloon 2406 and distal leg 2435. The distal leg 2435 is shown positioned over the channel extension 2416. The channel extension is shown positioned over a mandrel. The shaft 2404 is shown positioned distal to the distal leg 2435 and channel extension 2416 on the mandrel 2440. The working channel 2405 of the shaft 2404 is shown extending past an end of the shaft 2404. The camera support 2411 is shown coupled to the distal leg 2435.

Moving to FIG. 24B, the channel extension 2416 is shown mated to working channel 2405. The distal leg 2435 of the balloon 2406 (or, in some embodiments, an external thermoplastic layer) is shown positioned over the point at which the working channel 2405 and the channel extension 2416 meet.

FIG. 24C shows heat shrink 2442 applied over the location where the working channel 2405 and channel extension 2416 meet and where the distal leg 2435 extends. Upon application of heat, the layers melt together and couple the components.

FIG. 24D shows the working channel 2405 of the shaft 2404 laminated with the channel extension 2416 and the distal leg 2435 of the balloon 4206 (or external thermoplastic layer).

It will be appreciated that, in some embodiments, the components can be bonded using other methods (e.g., glue).

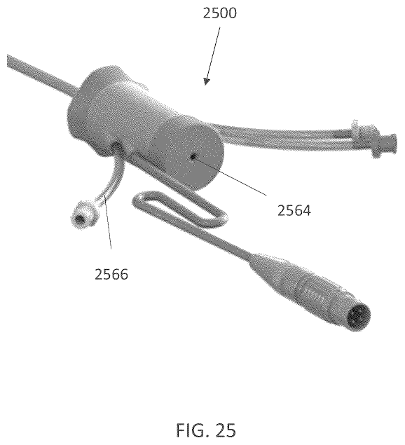

Moving to FIG. 25, an embodiment of a proximal end of the device 2500 is shown. A proximal entry point 2564 to the working channel is shown. The entry point 2564 can comprise a hemostatic seal. The device 2500 can comprise a fluid channel 2566 that can be used to flush and purge the working channel.

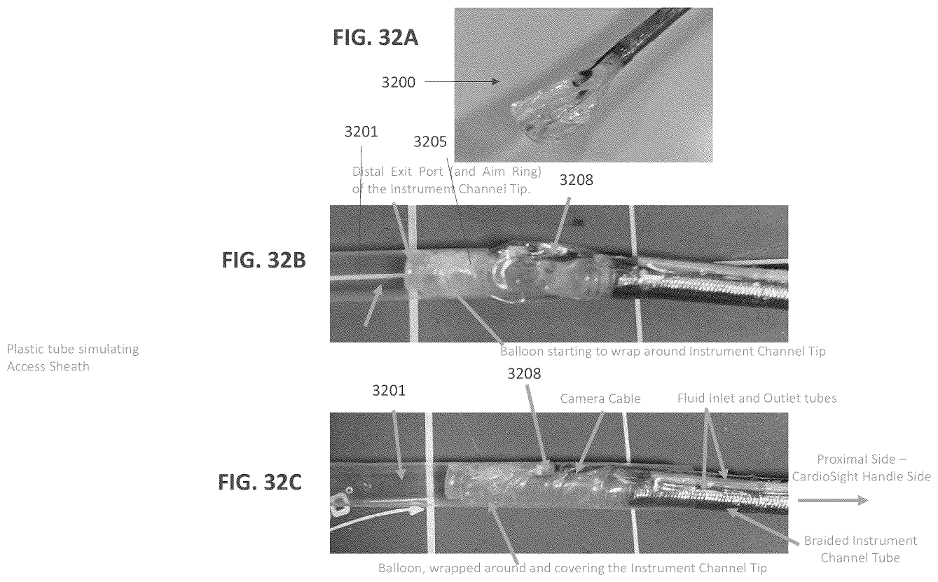

Referring to FIGS. 32A-32C, an embodiment of a method for deploying a balloon visualization module (BVM) from a stowed configuration is shown. Referring to FIG. 32A, a BVM 3200 in a deflated state is shown. The camera module connector 3202 is loose.

As the BVM 3200 is inserted into a sheath 3201 from its distal end, as shown in FIG. 32B, the balloon 3205 starts to wrap more closely around the channel extension 3206.

Once the BVM 3200 is fully inserted into the sheath 3201, the camera module 3208 moves into and becomes packed into the cut out portion of the channel extension.

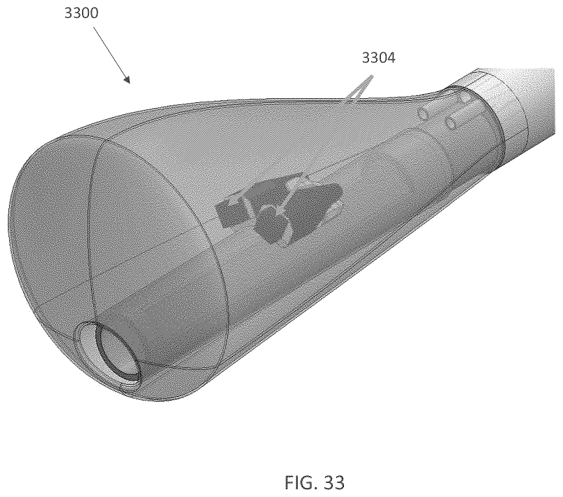

FIG. 33 shows an embodiment of a BVM 3300 comprising multiple cameras or imaging sensors 3304. Additional cameras or imaging sensors can operate outside the visible wavelength spectrum (e.g., ultraviolet or infrared) to provide additional information on examined structure (e.g., thermal imaging). A multiple camera embodiment can advantageously provide enhanced field of view, enhanced resolution, 3D imaging, and/or thermal imaging.

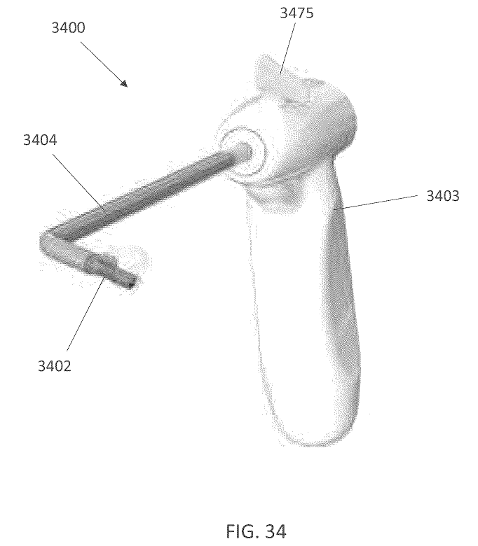

FIG. 34 provides an embodiment of an access device 3400 with a BVM 3402 as described herein. The device 3400 comprises a handle 3403. The sheath 3404 extends from the handle. The BVM 3402 and shaft extend through the sheath 3404. The handle 3403 comprises a control 3475 for affecting steering of the distal tip of the sheath.

It will be appreciated that the handle 3403 may comprise capabilities like those described with respect to device 2500 of FIG. 25 and the device of FIG. 27B.

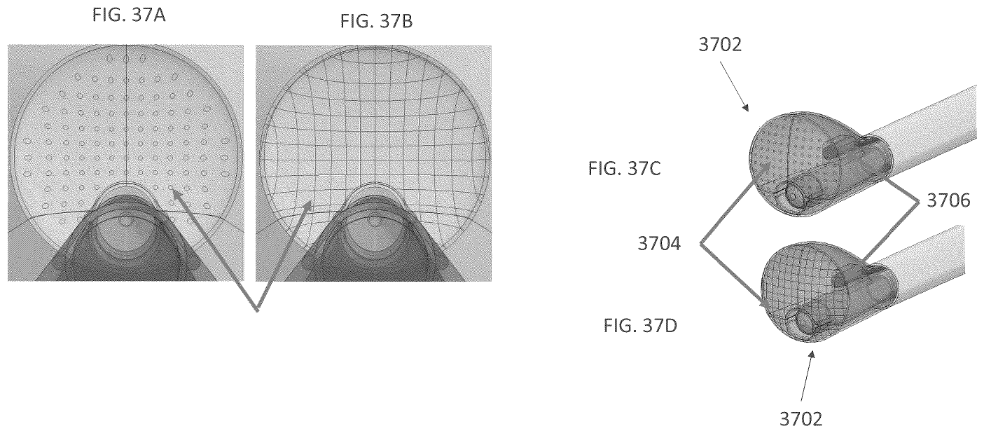

Moving to FIGS. 37A-37D, embodiments of balloons 3702 with a distal window 3704 comprising optical scales are provided. FIGS. 37A and 37C show the field of view from the camera 3706 and a perspective view of the BVM, respectively, of a BVM comprising a balloon with an optical scale comprising a point grid. FIGS. 37B and 37D show the field of view a perspective view of the BVM, respectively of a BVM comprising a balloon with an optical scale comprising cross hairs. Any other optical scale can also be used on the distal window of the balloon

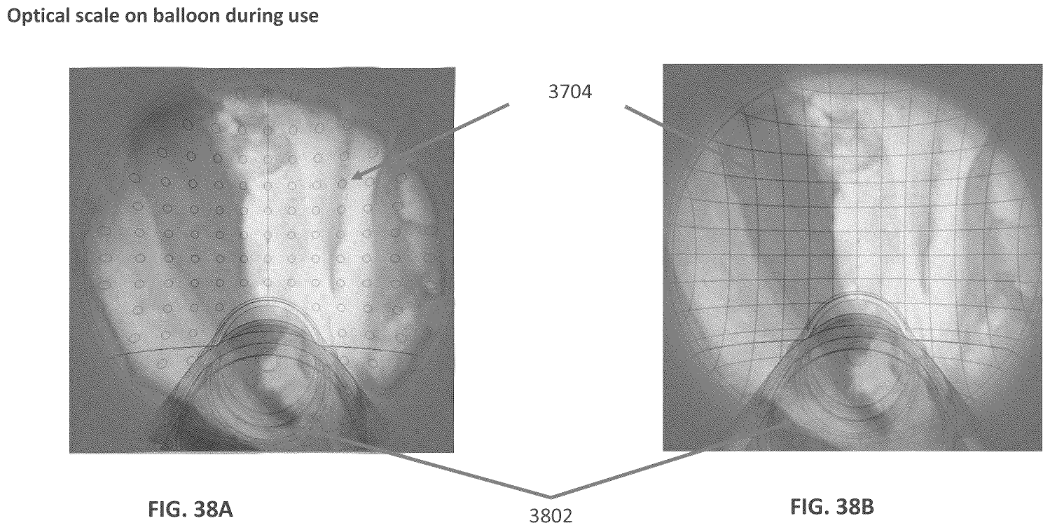

FIG. 38A shows the field of view from the camera module using a BVM as shown in FIGS. 37A and 37C. FIG. 38B shows the field of view from the camera module using a BVM as shown in FIGS. 37B and 37D. The scale allows precise positioning on targeted tissue and precise measurement of tissue structure sizes. The working channel 3802 (e.g., channel extension) is shown in a bottom portion of the field of view.

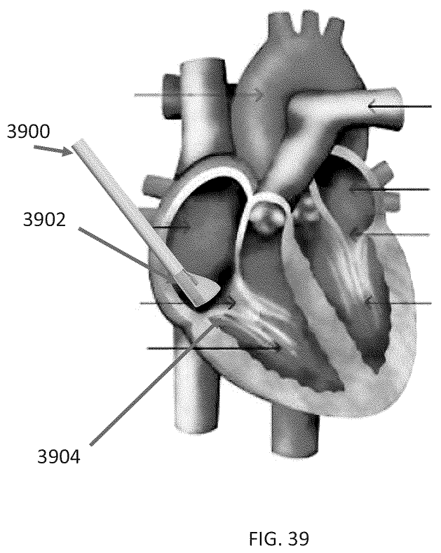

FIG. 39 shows an example of how the BVM can be used as a tissue manipulator. The device 3900 is shown positioned near the tricuspid valve. As shown in FIG. 39, the balloon 3902 can be used to gently fix or support moving (or floppy) structure while adequate position of a treatment or surgical tool or implant is found. In FIG. 39, the balloon 3902 is shown supporting moving or floppy tissue segments 3904 (e.g., tendons, valves, etc.).

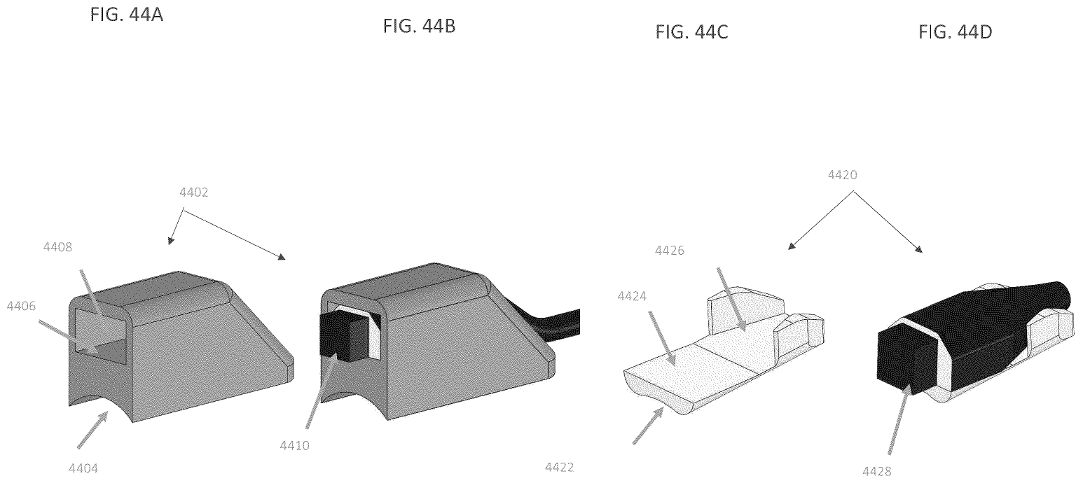

FIGS. 44A-44D show further embodiments of camera supports or mounts. The camera support can act as a position fixture for the camera. The position of the light source(s) of the camera module may or may not be fixed with the camera support.

The camera support can be configured to house one or more camera modules.

The camera support can comprise plastic (e.g., additive manufacturing, injection moulding, etc.). Other materials are also contemplated (e.g., metal, ceramic, silicone).

The camera support can be fixed to the balloon channel. In some embodiments, it fully surrounds the balloon channel.

FIG. 44A shows an embodiment of a camera support 4402. The support comprises a bottom surface 4404 configured to be joined to a device component (e.g., balloon channel). The surface 4404 can be contoured to better conform to the component to which it is to be joined.

The camera support further comprises a platform 4406. The camera module can rest upon the platform 4406.

In some embodiments, the camera support comprises a slot 4408. The camera module 4410 can be configured to be positioned within the slot 4408, as shown in FIG. 44B. The slot 4408 and/or platform 4406 can be sized to correspond to a size of the camera module.

FIGS. 44C and 44D show another embodiment of a camera support 4420. The support comprises a bottom surface 4422 configured to be joined to a device component (e.g., balloon channel). The surface 4422 can be contoured to better conform to the component to which it is to be joined.

The camera support further comprises a platform 4424. The camera module can rest upon the platform 4424.

In some embodiments, the camera support comprises a slot 4426. The camera module 4428 can be configured to be positioned within the slot 4426, as shown in FIG. 44B. The slot 4426 differs from the slot 4408 as it is open on the top such that a top portion of the camera module 4428 is exposed. The slot 4426 and/or platform 4424 can be sized to correspond to a size of the camera module.

The camera support can be coupled to the balloon channel (e.g., as described with respect to FIGS. 23A-23C. The camera module can then be placed within the camera support slot and bonded to the support (e.g., using glue).

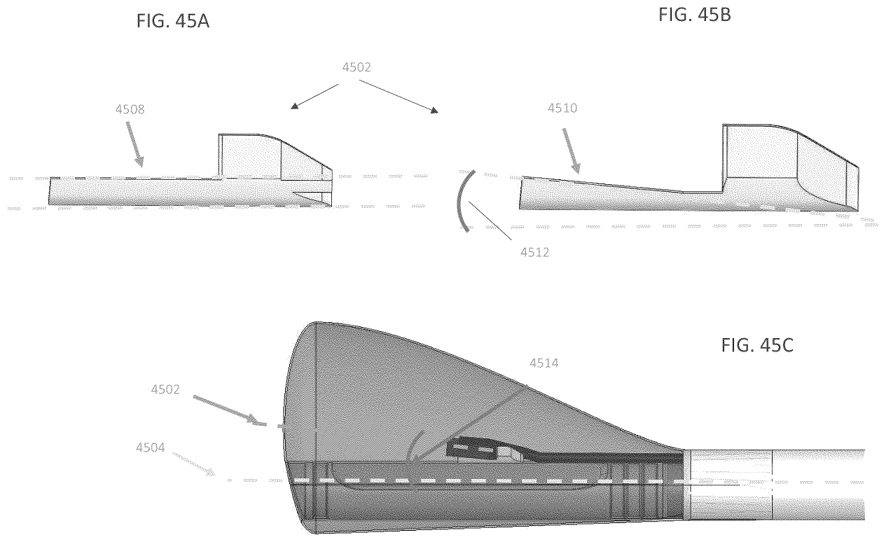

As shown in FIG. 45A-45C, the camera support 4502 can be used to set the center axis angle of the camera view relative to the longitudinal axis 4504 of the working channel/channel extension 4506. FIG. 45A shows a camera support 4502 with a parallel platform 4508. Such a camera support would place the center axis angle parallel to the longitudinal axis of the working channel/channel extension. FIG. 45B shows a camera support 4502 with an angled platform 4510. The angle 4512 of the platform places the camera view axis 4502 at an angle 4514 relative to the longitudinal axis 4504 of the working channel/channel extension. The angle 4512 of the platform can substantially correspond to the angle 4514 of the camera view axis relative to the longitudinal axis of the working channel/channel extension.

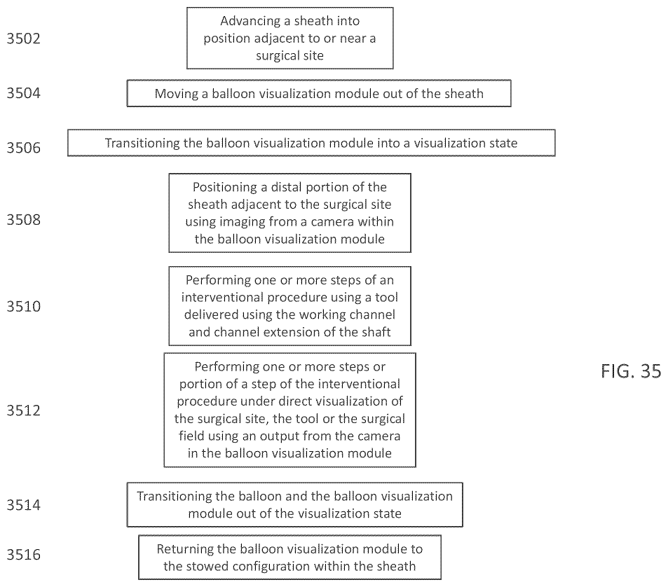

Referring now to FIG. 35, an embodiment of a method 3500 of using a BVM device is described. The method comprises the step 3502 of advancing an access sheath into position adjacent to or near a surgical site.

Step 3504 comprises moving the BVM device out of the sheath by advancing the shaft with the BVM coupled at its distal end relative to the access sheath.

The method comprises the step 3506 of transitioning the BVM into a visualization state. This step can comprise inflating the balloon, thereby moving the camera module into a desired position.

Step 3508 comprises positioning a distal portion of the device (e.g., the BVM) adjacent to the surgical site using imaging from a camera module within the BVM.

The method comprises the step 3510 of performing one or more steps of an interventional procedure using a tool delivered using the working channel and channel extension of the shaft.

Step 3512 comprises performing one or more steps of or portions of a step of the interventional procedure under direct visualization of the surgical site, the tool, or the surgical field using an output from the camera in the BVM.

The method comprises the step 3514 of transitioning the BVM out of the visualization state.

Step 3516 comprises retracting the BVM into the stowed configuration within the access sheath.

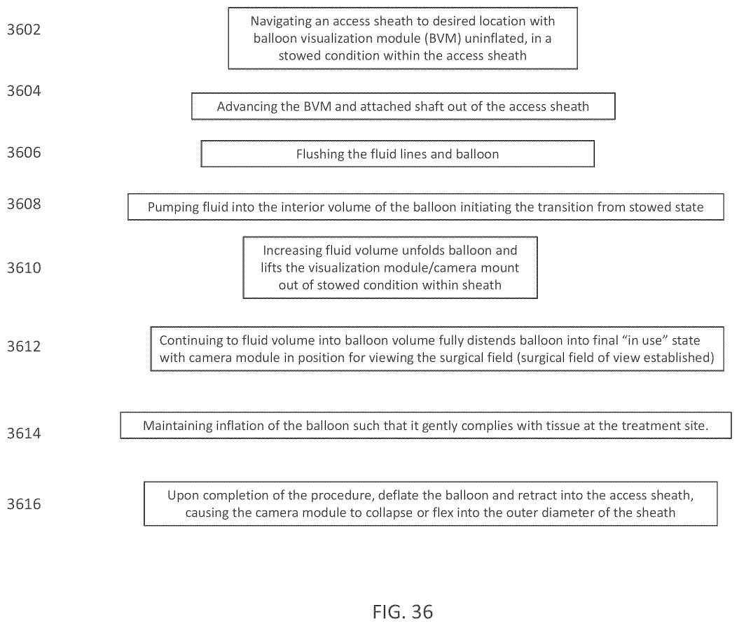

Moving to FIG. 36, an embodiment of a method of using a BVM device to perform an interventional surgical procedure is shown.

The method comprises the step 3602 of navigating the access sheath to a desired location with the BVM uninflated and in a stowed configuration within the access sheath.

Step 3604 comprises advancing the shaft comprising the BVM at its distal end out of the stowed configuration within the sheath.

The method comprises the step 3606 of flushing the fluid lines and balloon. Flushing can comprise continuously providing fluid to the fluid lines and the balloon.

Step 3608 comprises pumping fluid into the interior volume of the balloon initiating the transition to the inflated state.

The method comprises the step 3610 of increasing the fluid volume, causing the balloon to unfold/unfurl and lifting the camera module out of the stowed configuration.

Step 3612 comprises continuing to pump fluid into the balloon until it fully distends into an inflated state with the camera module in position for viewing the surgical field.

In some embodiments, visual feedback is used to determine completion of inflation. As soon as no major wrinkling is seen on the camera view and a clear field of view is present, the inflation is complete.

In some embodiments, a fixed volume can be used to determine complete inflation. The balloon can be prescribed to be inflated with a defined volume of fluid (e.g., saline).

In some embodiments, active pressure control can be used to determine complete inflation. The balloon pressure can be directly measured at a proximal end of the fluid lines. Based on the pressure data and a syringe pump connected to the inlet, a control loop can be constructed which can provide one or more of: constant balloon pressure, leak detection of the balloon, and contact force sensing with the tissue.

The method comprises step 3614, which comprises maintaining inflation of the balloon such that it gently complies with tissue at the treatment site during the procedure.

Step 3616 comprises, upon completion of the procedure, deflating the balloon and retracting it into the access sheath, causing the camera module to collapse or flex into the outer diameter of the sheath.

The sheath and BVM can then be withdrawn from the vasculature.

### Ablation (Atrial Fibrillation)

### Current State of the Art

Ablation can be surgical treatment for atrial fibrillation (AF). Atrial fibrillation is a worldwide cardiac problem, that is accompanied by the irregular rhythm of the heart (arrhythmia), and later increases the risk of stroke or heart failure. The visualization of the abnormal area, the surgical and ablation devices are necessary during the treatment. The basis of the treatment to create scar tissue on the abnormal site of the heart, with radiofrequency or cryothermal, so this area does not conduct the electrical signals of the heart, thereby sinus rhythm can be maintained. However, despite this treatment is often used, it is not risk free (e.g. bleeding, infection, heart valve damage).

### Clinical Issues to Solve

The treatment can be delivered with catheter (through the blood vessels, pulmonary vein isolation with radiofrequency or cryoballoon), or with minimally invasive video-assisted or completely thoracoscopic. During video-assisted thoracoscopic (VATS), a small camera, and surgical instruments are inserted into the chest through minimal incision(s). Other treatments are the Cox-Maze procedures (radiofrequency and cryothermal mixture) with minimally invasive right mini-thoracotomy (RMT) or as an addition to cardiac surgeries of other indication.

Accordingly, it is to be appreciated that in various embodiments the surgical access device, device shaft, working channel and balloon visualization module are adapted and configured for use in performing one or more steps of a minimally invasive, single port access, robotically assisted, interventional or surgical procedure to address the shortcomings of or clinical issues related to procedures for ablation (atrial fibrillation) along with, additionally or optionally, those related to improved visualization through application the BVM as well as instruments or devices provided utilizing the hybrid working channel.

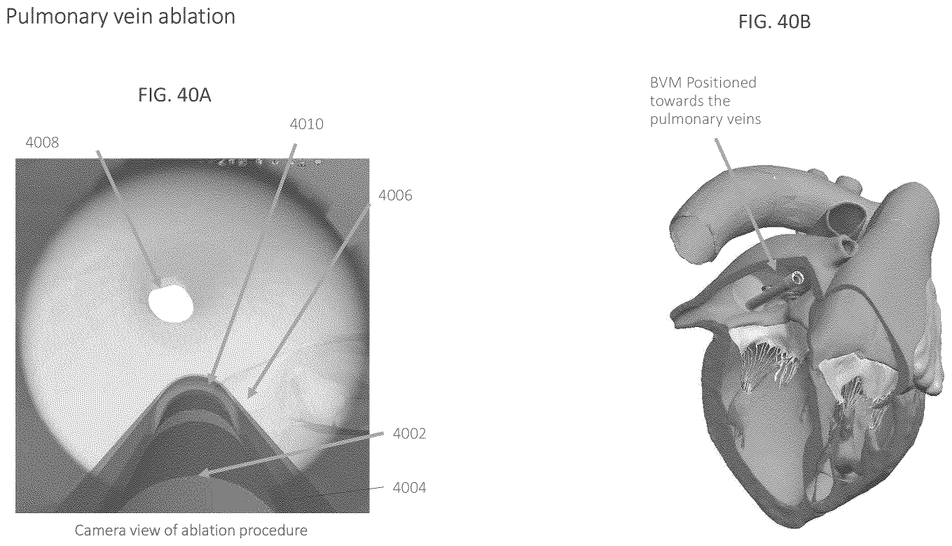

An exemplary method of using a BVM to perform pulmonary vein ablation follows. The device is set up in the left atrium according to the sequence described at, for example steps 3502-3508 of method 3500, described with respect to FIG. 35 or steps 3602-3614 of method 3600, described with respect to FIG. 36. The method further comprises maintaining visualization of the pulmonary vein entrances, as shown in FIG. 40B (which shows the BVM device positioned towards the pulmonary veins). An ablation electrode is advanced within the working channel of the device. A series of spot ablations is performed while positioning the ablation electrode using the output from the camera module of the BVM. Visual verification of whether a contiguous coagulated zone is created around the vein entrance using output from the camera module of the BVM is performed. The ablation electrode is retracted from the working channel of the BVM shaft.

The steps 3514, 3516 of method 3500, described with respect to FIG. 35 or steps 3616 of method 3600, described with respect to FIG. 36, can then be performed and the sheath retracted from the vasculature.

FIG. 40A shows a field of view from the camera module during an ablation procedure. An ablation electrode 4002 is shown within the working channel 4004 (e.g., channel extension). The pulmonary vein 4008 and a previously ablated surface 4006 are visible within the field of view. The target site 4010 for the next spot ablation to provide a continuous ablation line around the vein entry is shown at a distal end of the channel 4004.

### LAA Occlusion

### Current State of the Art

Left atrial appendage (LAA) is a pouch in the left atrial wall of the heart, with highly variable anatomy. Outside it can be round-, triangle-, waterdrop-shaped, and inside it can be chicken wing, cactus, windsock, or cauliflower morphology. In the case of atrial fibrillation (AF), the LAA contributes blood clot formation and increase the risk of stroke, thereby it needs to be treated. Currently there are two methods, the LAA occlusion (LAAO) and the LAA exclusion (LAAE).

### Clinical Issues to Solve

Because the LAA have a wide variety of anatomy, the treatment is also complex, therefore, it is necessary to visualize the formation before or during the treatment.

During LAAO, catheter-based devices, with lobe or umbrella ends, inserted endovascularly into the LAA, to block the blood flow. Usually, preprocedural imaging is used before the procedure. 2D or 3D transesophageal echocardiography (TOE) is a gold-standard, other procedure is the cardiac computed tomography angiography (CCTA). However, the 3D procedure shows a more accurate picture of the LAA anatomy, than the 2D, but the temporal resolution is much slower. Another imaging during the treatment, is the Fluoroscopic 2D imaging, but it does not provide sufficient anatomic detail or guidance to visualize the planned structural heart intervention.

LAA closure can be performed surgically, either as a concomitant procedure during open-heart surgery or as a stand-alone surgical procedure as part of minimally invasive (mini-thoracotomy or thoracoscopy) arrhythmia surgery. High failure rates present and no robust outcome data available.

Accordingly, it is to be appreciated that in various embodiments the surgical access device, device shaft, working channel and balloon visualization module are adapted and configured for use in performing one or more steps of a minimally invasive, single port access, robotically assisted, interventional or surgical procedure to address the shortcomings of or clinical issues related to procedures for left atrial appendage (LAA)/left atrial occlusion along with, additionally or optionally, those related to improved visualization through application the BVM as well as instruments or devices provided utilizing the hybrid working channel.

### Visualization During Minimally Invasive Mitral Surgery

### Current State of the Art

The minimally invasive cardiac surgery (MICS) more and more popular among cardiac surgeons, because this process has many advantages. The advantages are less pain, bleeding and risk of wound infection, smaller incisions thereby faster recovery, better cosmetic result, shorter hospital stay.

During minimally invasive mitral valve surgery (MIMVS), a minimally surgical incision must be made, with right mini thoracotomy, which enables the treatment of mitral disorders, like mitral valve regurgitation or stenosis. Mitral valve regurgitation is when the valve does not close properly, thereby allowing blood to leak between the chambers. In contrast, during mitral stenosis, the mitral valve does not open enough, causing congestion, arrhythmia (atrial fibrillation), pulmonary hypertension, ultimately right ventricular failure.

One of the treatment options for mitral stenosis or regurgitation surgery is to replace the valve with a biological (pig, cow, or human heart tissue) or mechanical valve. Before the operation, it is necessary to map the patient's heart. This can be done with electrocardiogram (ECG), chest X-ray, transesophageal echocardiogram (TEE) or coronary angiogram. During the procedure, a heart-lung machine (CPB) is used, by inserting cannulas into the artery and vein. Before and after the replacement, a transesophageal echocardiogram (TEE) is used, will be inserted through the esophagus.

### Clinical Issues to Solve

The surgery is performed with minimally invasive thoracotomy and visualization is performed using a thoracoscope.

A direct, and appropriate visualization, during the surgery, is exceptionally helpful for the doctors. Accordingly, it is to be appreciated that in various embodiments the surgical access device, device shaft, working channel and balloon visualization module are adapted and configured for use in performing one or more steps of a minimally invasive, single port access, robotically assisted, interventional or surgical procedure to address the shortcomings of or clinical issues related to procedures for minimally invasive mitral surgery along with, additionally or optionally, those related to improved visualization through application the BVM as well as instruments or devices provided utilizing the hybrid working channel.

### Visualization During Minimally Invasive Aortic Surgery

### Current State of the Art

The minimally invasive cardiac surgery (MICS) more and more popular among cardiac surgeons, because this process has many advantages. The advantages are less pain, bleeding and risk of wound infection, smaller incisions thereby faster recovery, better cosmetic result, shorter hospital stay.

During minimally invasive aortic valve surgery (MIAVS), a minimally surgical incision must be made, which enables the treatment of aortic disorders, through aortic valve replacement or aortic valve repair. In the case of aortic stenosis, the valve is narrow, calcified, thereby not allowing the left ventricle to work properly, causing left ventricular hypertrophy, and, in the case of aortic regurgitation, the valve does not close properly, so the blood flows back into the left ventricle, thereby causing left ventricular hypertrophy also.

Before the operation, it is necessary to map the patient's heart and aorta. This can be done with electrocardiogram (ECG), computed tomography (CT), transesophageal echocardiogram (TEE), similar to mitral valve replacement. During the procedure, a heart-lung machine (CPB) is used, by inserting cannulas into the artery and vein. Before and after the replacement, a transesophageal echocardiogram (TEE) is used, will be inserted through the esophagus.

### Clinical Issues to Solve

The surgery, nowadays, is performed with right anterior thoracotomy (RAT) or ministernotomy (MS) and visualization is performed using a thoracoscope.

A direct, and appropriate visualization, during the surgery, is exceptionally helpful for the doctors. Accordingly, it is to be appreciated that in various embodiments the surgical access device, device shaft, working channel and balloon visualization module are adapted and configured for use in performing one or more steps of a minimally invasive, single port access, robotically assisted, interventional or surgical procedure to address the shortcomings of or clinical issues related to procedures for minimally invasive aortic surgery along with, additionally or optionally, those related to improved visualization through application the BVM as well as instruments or devices provided utilizing the hybrid working channel.

### Visualization During Coronary Surgery

### Current State of the Art

During coronary heart disease, the walls of the coronary arteries (carry blood to the heart) become clogged, due to fatty deposits.

The primary treatment is the coronary artery bypass graft (CABG), which can now be performed with minimally invasive surgery (MIDCAB). During the operation, a bypass is made to the vascular area behind the stenosis, for which a graft is used. The coronary angiography was the primary mode of presentation, clinically. Contrast is injected into the coronary arteries to identify existing blockages or stenosis caused by plaques/calcifications.

### Clinical Issues to Solve

A direct, and appropriate visualization, during the surgery, is exceptionally helpful for the doctors. Accordingly, it is to be appreciated that in various embodiments the surgical access device, device shaft, working channel and balloon visualization module are adapted and configured for use in performing one or more steps of a minimally invasive, single port access, robotically assisted, interventional or surgical procedure to address the shortcomings of or clinical issues related to procedures for minimally invasive coronary surgery along with, additionally or optionally, those related to improved visualization through application the BVM as well as instruments or devices provided utilizing the hybrid working channel. Visualization During Minimally Invasive Surgery for Intrapericardial Tumors

### Current State of the Art

Intrapericardial tumours (or teratomas) are abnormal tissue proliferations, inside the heart. The intrapericardial tumours are rare heart tumors that are usually diagnosed in infants and newborns. Usually, these are benign tumors, but they can also be life-threatening.

### Clinical Issues to Solve

In this case, to remove the tumours, open heart surgery is inevitable. Two-dimensional echocardiography is considered the best diagnostic imaging method for tumor visibility, but magnetic resonance imaging (MRI) may also be used (it may have the advantage that neighboring areas may also be better see.

A direct, and appropriate visualization, during the surgery, is exceptionally helpful for the doctors. Accordingly, it is to be appreciated that in various embodiments the surgical access device, device shaft, working channel and balloon visualization module are adapted and configured for use in performing one or more steps of a minimally invasive, single port access, robotically assisted, interventional or surgical procedure to address the shortcomings of or clinical issues related to procedures for minimally invasive surgery for intrapericardial tumours along with, additionally or optionally, those related to improved visualization through application the BVM as well as instruments or devices provided utilizing the hybrid working channel.

In still further alternative embodiments, the various embodiments the surgical access device, device shaft, working channel and balloon visualization module are adapted and configured for use in via transjugular access for use in performing one or more steps of a minimally invasive, single port access, robotically assisted, interventional or surgical procedure to address the shortcomings of or clinical issues as detailed herein. The surgical access device may also include embodiment specific to a transjugular access medical procedure. Additionally or optionally, there may be other modifications of improvements related to improved visualization through application the BVM as well as instruments or devices provided utilizing the hybrid working channel. By way of example and not limitation, exemplary transjugular procedures may include: pacemaker lead removal, endomyocardial biopsy, transcatheter mitral valve replacement/repair and transcatheter tricuspid valve replacement/repair.

### Pacemaker Lead Removal

### Current State-of-the-Art

Pacemaker lead implantation initiates a fibrous growth process that usually results in lead-vascular binding sites along the vascular path and the electrode-myocardial interface. Removal of adhered or perforated leads can result in significant complications, such as superior vena cava tear, cardiac avulsion, and even death. Imaging is critical in defining potential vascular adhesions, cardiac perforations and any aberrant lead course, and modifying the approach to the specific challenges of the case.

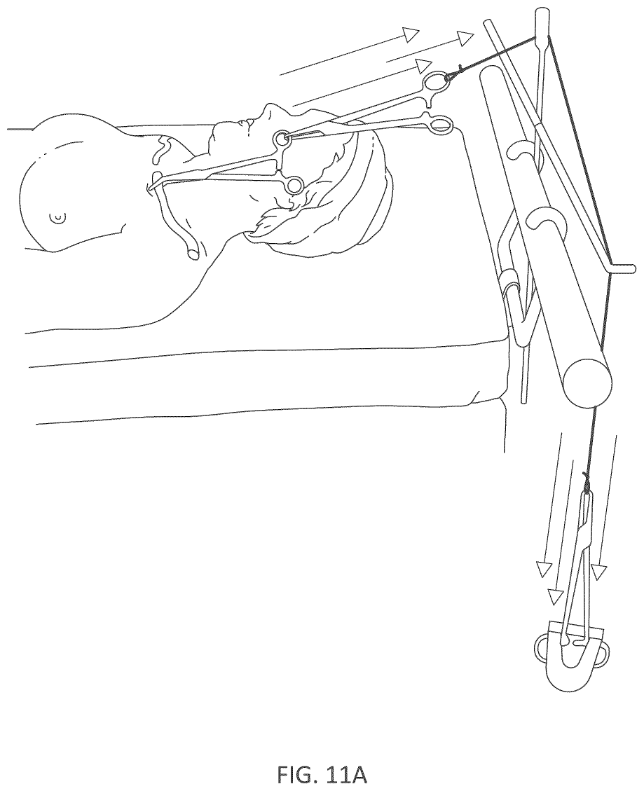

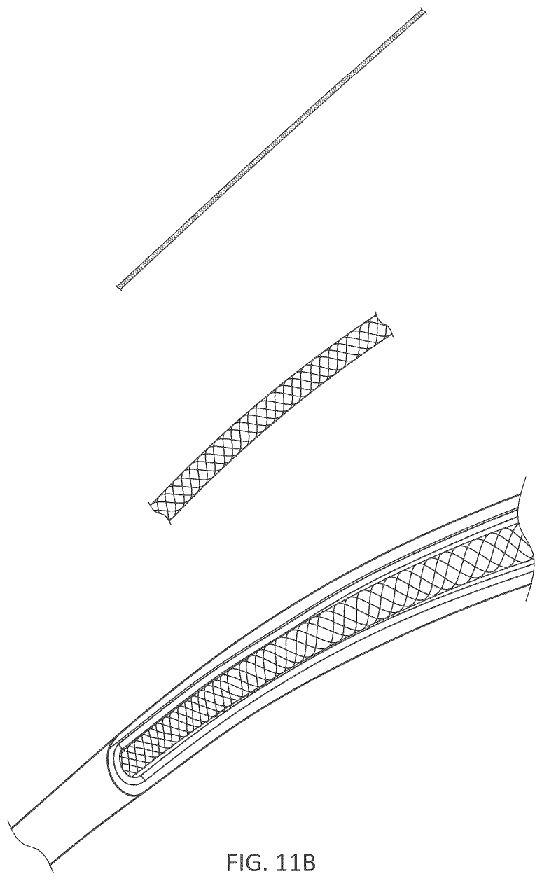

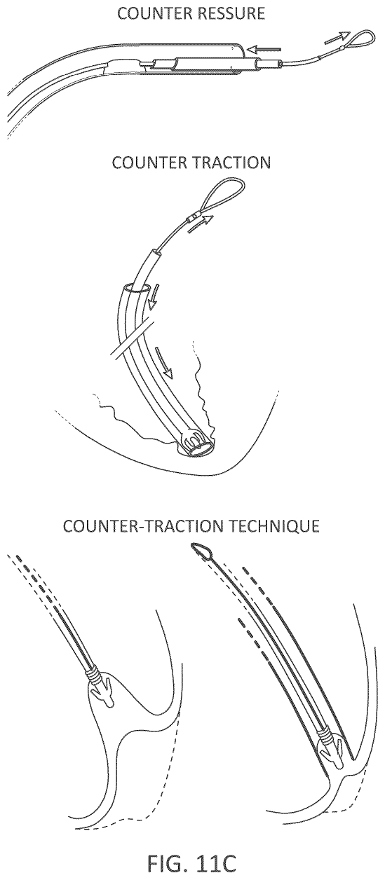

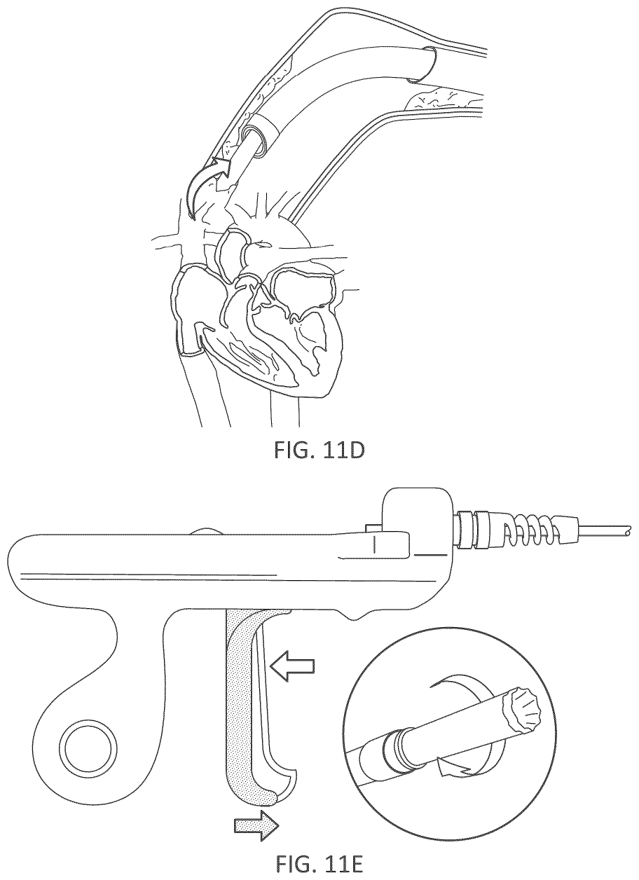

Referring to FIGS. 11A-11D, conventional pacemaker lead removal techniques are shown. These techniques include removal by force, removal by force using locking stylet, use of a counter traction sheath, and removal by resection using mechanical and laser cutter sheaths. These techniques all present challenges and potential risks.

During removal by force, an embodiment of which is shown in FIG. 11A, force is applied to the end of the lead to apply constant outward traction to facilitate lead removal. This method may disadvantageously cause lead disintegration or breaking during the procedure. This method may also result in uncontrolled tearing of the scar tissue around the peacemaker lead which can lead to complications.

FIG. 11B shows a locking stylet that is conventionally used for pacemaker lead removal. Removal by force using a locking stylet can include inserting and deploying a locking stylet inside the lead lumen. The stylet provides even pulling force along the axis of the lead which can reduce the chance of the lead breaking. This method can lead to uncontrolled tearing of the scar tissue around the pacemaker lead which can lead to complications.

When using a counter traction sheath for lead removal, as shown in FIGS. 11C and 11D, it can be placed around the pacemaker lead to support the surrounding tissue prior to pulling the electrode. Using this method can lead to uncontrolled tearing of the tissue, though the tearing may be limited by the size of the sheath.

Removal by resection using mechanical (FIG. 11E) and laser cutter sheaths (FIG. 11F) can comprise adding cutting elements to the distal end of a counter traction sheath. This removal method presents a likelihood of piercing the heart or vein tissue as there can be very limited feedback on the cutting action of the devices.

As described, conventional pacemaker removal techniques provide a number of challenges including severity of ingrowth, inability to examine scar tissue formation around pacemaker electrode, lack of visual feedback during procedure, lack of feedback on forces directly applied to the tissue during procedure. Generally, the electrode is separated from the scar tissue in a highly uncontrolled manner without any visualization.

### Clinical Issues to Solve

Procedural imaging has two purposes: to further exploration of any concerns suggested by preprocedural imaging and to monitor the patient during the procedure.

In still further alternative embodiments, the various embodiments the surgical access device, device shaft, working channel and balloon visualization module are adapted and configured for use in via transjugular access for use in performing one or more steps of a pacemaker lead removal procedure to address the shortcomings of or clinical issues as detailed herein. The surgical access device may also include embodiments specific to a transjugular access medical procedure. Additionally or optionally, there may be other modifications of improvements related to improved visualization through application the BVM as well as instruments or devices provided utilizing the hybrid working channel so as to provide a platform for access and direct visualization to guide instruments into the beating heart. In some aspects, the inventive surgical access device may provide near-field, direct visualization of target cardiac tissues and instrument/tissue interaction. Additionally or alternatively, embodiments may be developed based on a better understanding of cardiac anatomy and adherences to guide lead extraction procedure and also to a faster detection and close monitoring of potential complications as a result of the beneficial use of the embodiments of the BVM.

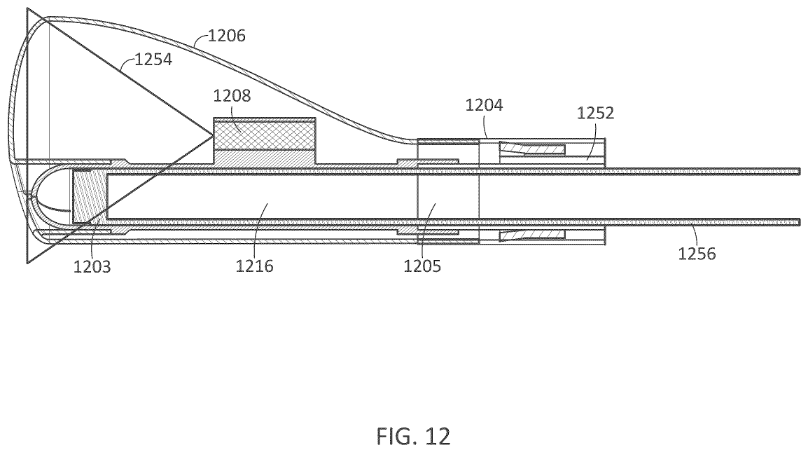

Referring now to FIG. 12, another embodiment of an access device 1200 with a BVM 1202 is shown in a deployed configuration. Unless described otherwise, the device 1200 can comprise features similar to those described elsewhere herein (e.g., with respect to device 400, 500, 600, 700, 800, 900, etc.). The device 1200 comprises a cutting element 1203 (e.g., positioned in an instrument channel of the BVM) in addition to the BVM 1202.

The device 1200 comprises a sheath 1204. A working channel 1205 extends through the sheath. A flexible extension 1216 is coupled to the sheath 1204 at its distal end and extends the working channel 1205.

The cutting element 1203 is positioned at or near a distal end of the flexible extension 1216. The cutting element 1203 can be positioned such that it is always or generally in the field of view 1254 of the camera module 1208. In some embodiments, the cutting element comprises a mechanical cutter, an RF electrode, a laser cutter.

A cutting element actuation member 1256 can extend from the cutting element 1203 along the sheath proximally towards the handle. It will be appreciated that the handle can comprise control mechanisms used to actuate the cutting element 1203.

The device 1200 can comprise a pressure sensing lumen 1252 configured to provide data on tissue contact forces. Other pressure sensing configurations are also possible. In some embodiments, balloon pressure can be directly measured at a proximal ends of the fluid lines. Based on the pressure data and a pressure source (e.g., syringe pump) connected to the inlet of the balloon, a control loop can be constructed which can provide one or a combination of: constant balloon pressure, leak detection of the balloon, and contact force sensing with the tissue.

The balloon comprises an asymmetric bulb shape with the bulk of its volume positioned above the sheath 1204 and working channel 1205. The balloon can increase in diameter from its proximal end to its distal end. The balloon comprises a curved distal end providing the distal window for the camera module 1208. The distal window 1220 can be sized and shaped to approximate a desired field of view for the camera/lighting/imaging module 1208.

A proximal end of the balloon can be generally tubular shaped with a generally circular cross section to correspond to a distal end of the sheath 1204.

The balloon is configured with a channel 1222 shaped to accommodate a distal end of the flexible channel extension 1216. The balloon 1206 can be coupled to the flexible channel extension 616 around the channel 1222.

As described above, a first portion of a distal end of the balloon is shaped to provide alignment and access to a working channel 1205 within the sheath 1204 and a second portion of the distal end of the balloon is sized and shaped to correspond to a desired field of view for the camera/lighting/imaging module 1208.

An inlet channel 1210 and outlet channel 1212 extend along the shaft 1204 and are in fluid communication with the balloon 1206.

In some embodiments, the camera module 1208 is positioned on a top portion of the flexible channel extension 1216.

In some embodiments, the balloon comprises silicone. Other materials (e.g., Pebax®, Nylon, Polyester/PET, special compounded blends such as TPU/Pebax® and Pebax®/nylon, multilayer structures, etc.) are also contemplated.

In some embodiments, a durometer of the material can be about shore 80-90 A (e.g., shore85 A). Other durometers (e.g., shore 20-60 A, 30-40 A, 35-45 A, 45-75 A, 75-85 A, 60-100 A, 70-90 A, etc.) are also contemplated,

The balloon can comprise a thickness of about 0.20-0.25 mm (or about 0.15-0.30 mm, or about 0.10-0.35 mm, etc.).

In some embodiments, the balloon comprises a uniform thickness. In some embodiments, the thickness of the balloon varies.

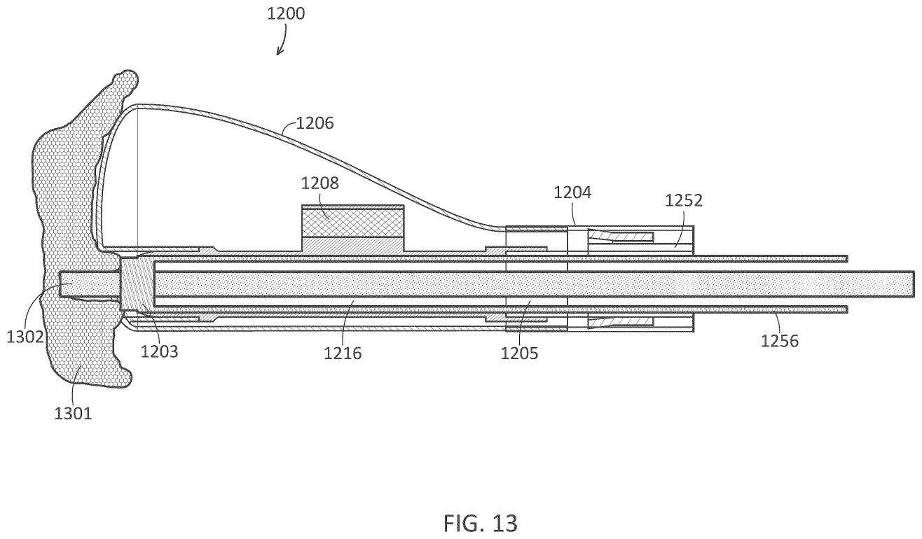

FIG. 13 shows a cross section side view of the device 1200 in its deployed configuration in position to remove a pacemaker lead. The device can act as a counter traction sheath with the balloon acting a visualization tool, and configured to exclude blood from the field of view of the camera. The balloon can alsoact as a manipulator to support tissue around the ingrown area while the pacemaker electrode is pulled.

The cutting element 1203 provides controlled separation of the lead electrode from the scar tissue. The cutting element can comprise a mechanical cutter, an RF electrode, a laser cutter. Other technologies are also contemplated.

In some embodiments, the balloon comprises a pressure sensor or pressure sensing lumen to provide data on tissue contact forces.

The principle of operation can be similar to or the same as the conventional method using a counter traction sheath with cutting elements. The device is placed around the pacemaker lead and then advanced. The lead electrode is then separated from the scar tissue by activating the cutting element under direct visualization, and with known contact forces.

An exemplary method of using a BVM to perform pacemaker lead removal follows. The device is set up at the right ventricle according to the sequence described at, for example steps 3502-3508 of method 3500, described with respect to FIG. 35 or steps 3602-3614 of method 3600, described with respect to FIG. 36. The device can be navigated to the targeted pacemaker lead using output from the camera module. Scar tissue formation around the pacemaker lead is inspected using output from the camera module. Optionally, tissue manipulating devices (e.g., forceps, blades, etc.) can be advanced within the working channel of the device to free the pacemaker lead from the scar tissue. Once the pacemaker lead is removed, the tissue manipulating devices can be retracted from the working channel. The steps 3514, 3516 of method 3500, described with respect to FIG. 35 or steps 3616 of method 3600, described with respect to FIG. 36, can then be performed and the sheath retracted from the vasculature.

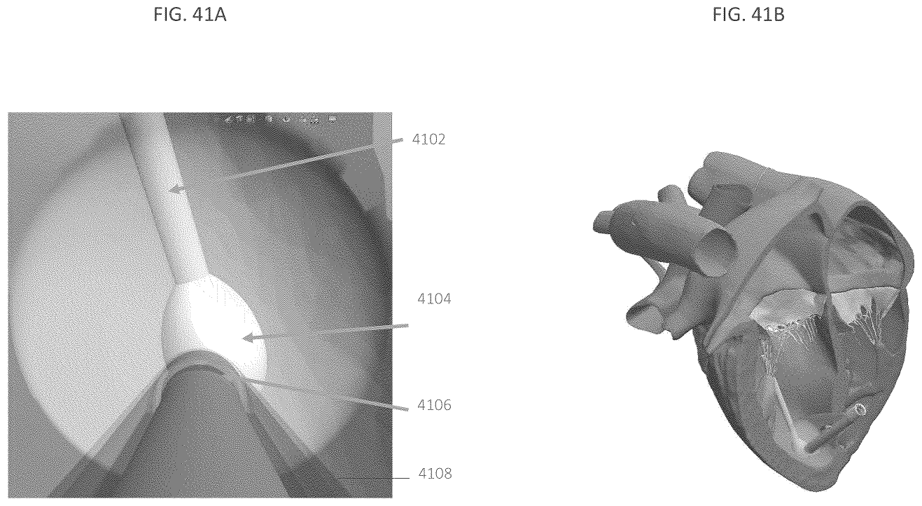

FIG. 41A shows the field of view from a camera module showing the pacemaker lead 4102 and scar tissue formation 4104 at the anchor portion of the pacemaker lead. The cutting element 4106 (e.g., blade, biopsy forceps, etc.) is shown within the working channel 4108 (e.g., channel extension). The cutting element 4106 can be used to free the pacemaker lead.

FIG. 41B shows the BVM visualizing the scarred lead from the side.

The BVM device can be used as an inspectional, diagnostic tool to assist conventional pacemaker lead removal techniques. Surgery can optionally be performed with a cutting element delivered through the working channel. The cutting element can be mechanical or RF powered.

A method pacemaker lead removal over the wire follows. A targeted pacemaker lead can be inserted into the distal exit of the working channel. The BVM device is advanced as the pacemaker lead serves as a guidewire. While the BVM is packed within the access sheath with the camera module bent into the working channel, there is still sufficient space for a 1.5-2 mm outer diameter pacemaker lead to pass through. Once the device is within a large enough cavity, the BVM is deployed (e.g., according to steps 3504-3508 of method 3500 described with respect to FIG. 35 or steps 3604-3612 of method 3600 described with respect to FIG. 36). Scar tissue formation is inspected around the pacemaker lead using output from the camera module. Optionally, a cutting device can be advanced within the working channel around the pacemaker lead to free the pacemaker lead from the scar tissue under visual control from the camera module. Once the pacemaker lead is removed, the tissue manipulating devices can be retracted from the working channel. The steps 3514, 3516 of method 3500, described with respect to FIG. 35 or steps 3616 of method 3600, described with respect to FIG. 36, can then be performed and the sheath retracted from the vasculature.

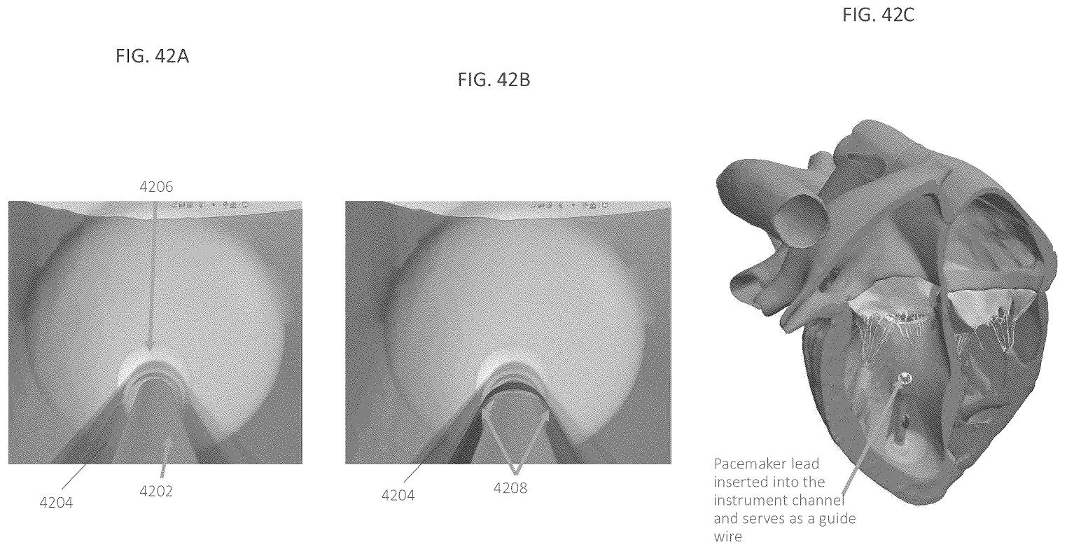

FIG. 42A shows a field of view from the camera module of a BVM device with a pacemaker lead 4202 within the working channel 4204. The scarred pacemaker lead anchor 4206 is shown. FIG. 42B shows a circular cutting element 4208 delivered through the working channel 4204.

FIG. 42C shows a view of the pacemaker lead inserted into the working channel of the BVM device and serving as a guide wire.

This method using the BVM device can assist the conventional pulling method. As the electrode gets pulled, the balloon gently holds back surrounding tissues while tissue strain can be visually inspected.

The cutting element can be mechanical or active RF powered.

### Endomyocardial Biopsy (Myocarditis, Heart Transplantation)

### Current State of the Art

Endomyocardial biopsy (EMB) is an invasive clinical procedure, that is used to sample myocardium for histological analysis and therefore, rejection after heart transplantation, cardiomyopathies, myocarditis, drug toxicities can be diagnosed.

### Clinical Issues to Solve

During EMB, transthoracic echocardiogram (TTE) is used, due to control of the flexible bioptome and avoid possible damage. Endomyocardial biopsy is not completely risk-free (cardiac tamponade).

In still further alternative embodiments, the various embodiments the surgical access device, device shaft, working channel and balloon visualization module are adapted and configured for use in via transjugular access for use in performing one or more steps of an Endomyocardial biopsy (myocarditis, heart transplantation) procedure to address the shortcomings of or clinical issues as detailed herein. The surgical access device may also include embodiments specific to a transjugular access medical procedure. Additionally or optionally, there may be other modifications of improvements related to improved visualization through application the BVM as well as instruments or devices provided utilizing the hybrid working channel so as to provide a platform for access and direct visualization to guide instruments for use in these procedures.

An exemplary method of using a BVM to perform a biopsy follows. The device is set up at or near the surgical site according to the sequence described at, for example steps 3502-3508 of method 3500, described with respect to FIG. 35 or steps 3602-3614 of method 3600, described with respect to FIG. 36. Biopsy forceps are advanced within the working channel and a biopsy is performed. The biopsy forceps are retracted from the working channel.

The steps 3514, 3516 of method 3500, described with respect to FIG. 35 or steps 3616 of method 3600, described with respect to FIG. 36, can then be performed and the sheath retracted from the vasculature.

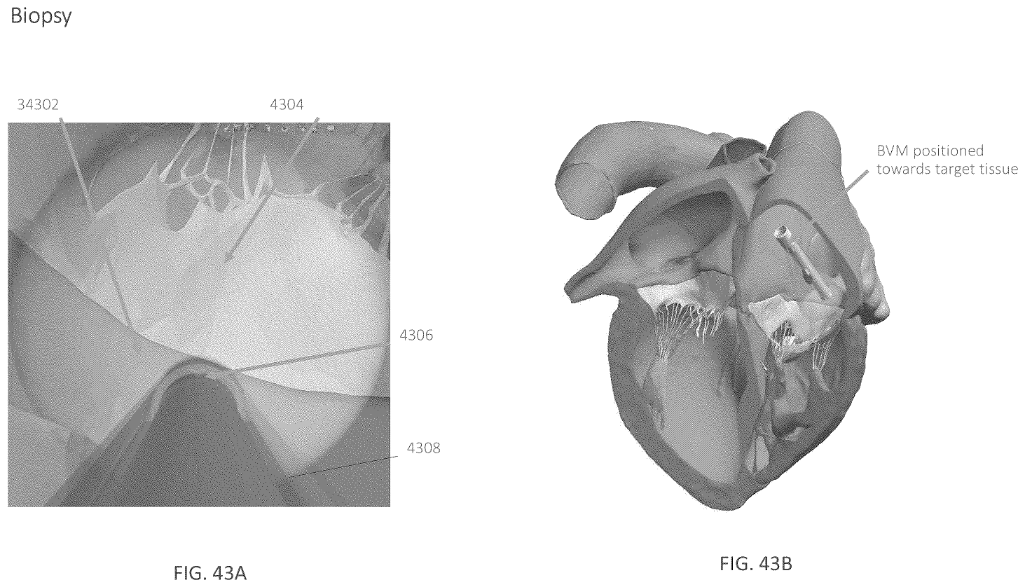

FIG. 43A shows a view from a camera module of BVM device performing a biopsy procedure at an annulus of a valve 4302. The annulus 4302 and valve leaflets 4304 are visible within the field of view. Biopsy forceps 4306 are positioned within the working channel 4306 (e.g., channel extension).

FIG. 43B shows the BVM device positioned toward the target tissue.

### Plication Clip for Mitral Valve

### Current State of the Art

For the treatment of abnormally functioning mitral valves (primary mitral regurgitation MR): degenerative MV disease resulting in anatomical changes in the valve and chordal that cause MR, secondary MR: ischemic or non-ischemic left ventricular failure with an enlarged mitral annulus, or dilatation of the left atrium in atrial fibrillation): minimally invasive mitral valve clips are used. These are tiny, metal clips, that are delivered by transcatheter approach to the mitral valve. This is called a Transcatheter Mitral Valve Repair (TMVr), but there are also options to a complete artificial valve delivery: Transcatheter Mitral Valve Replacement (TMVR).

### Clinical Issues to Solve

### Transcatheter Mitral Valve Repair (TMVr)

There are different methods of treatment, for example, transcatheter edge-to-edge repair (TEER-MitraClip, Pascal), direct/indirect annuloplasty (Cardioband, Mitralign, Carillon), and chordal repair (NeoChord). They can be used for both primary and secondary MR, with the exception of annuloplasty, which is for secondary MR only.

In still further alternative embodiments, the various embodiments the surgical access device, device shaft, working channel and balloon visualization module are adapted and configured for use in via transjugular access for use in performing one or more steps of an Plication clip for mitral valve procedure to address the shortcomings of or clinical issues as detailed herein. The surgical access device may also include embodiments specific to a transjugular access medical procedure. Additionally or optionally, there may be other modifications of improvements related to improved visualization through application the BVM as well as instruments or devices provided utilizing the hybrid working channel so as to provide a platform for access and direct visualization to guide instruments for use in these procedures. Transcatheter Mitral Valve Replacement (TMVR)

There are several different approaches, such as transseptal (HighLife TMVR System, Sapien M3 System, EVOQUE TMVR System) and transapical (Tendyne Mitral Valve System, Tiara TMVR System, Intreped TMVR System). TMVR clinical experience involved the following three main conditions: valve-in-valve, valve-in-ring, valve-in-native ring.

In still further alternative embodiments, the various embodiments the surgical access device, device shaft, working channel and balloon visualization module are adapted and configured for use in via transjugular access for use in performing one or more steps of a Transcatheter Mitral Valve Replacement (TMVR) procedure to address the shortcomings of or clinical issues as detailed herein. The surgical access device may also include embodiments specific to a transjugular access medical procedure. Additionally or optionally, there may be other modifications of improvements related to improved visualization through application the BVM as well as instruments or devices provided utilizing the hybrid working channel so as to provide a platform for access and direct visualization to guide instruments for use in these procedures.

### Visualization During MitraClip Procedure

### Current State of the Art

One of the treatment options for mitral valve repair is MitraClip, which is based on the traditional edge-to-edge method. It has 2 main components: metal clip coated in polyester fabric and the catheter. One of the most reliable treatments, but rarely some complications may occur (atrial septal defect, bleeding, pericardial effusion, endocarditis, clip detachment, clip embolization, mitral stenosis).

### Clinical Issues to Solve

The device is delivered to the left atrium through a catheter inserted on a leg vein. Transesophageal echocardiography (TEE) and fluoroscopy may be applied to visualization during treatment.

In still further alternative embodiments, the various embodiments the surgical access device, device shaft, working channel and balloon visualization module are adapted and configured for use in via transjugular access for use in performing one or more steps of a Visualization during MitraClip procedure to address the shortcomings of or clinical issues as detailed herein. The surgical access device may also include embodiments specific to a transjugular access medical procedure. Additionally or optionally, there may be other modifications of improvements related to improved visualization through application the BVM as well as instruments or devices provided utilizing the hybrid working channel so as to provide a platform for access and direct visualization to guide instruments for use in these procedures.

### Plication Clip for Tricuspid Valve

### Current State of the Art

Tricuspid regurgitation is a very common heart valve disease. The tricuspid valve is located between the right atrium and ventricle, if it does not close well the blood flows backwards from the right ventricle to the right atrium, increases the risk of cardiovascular morbidity and mortality on the long term.

### Clinical Issues to Solve

TV intervention devices are: coaptation and leaflet devices (TriClip, Pascal, Mistral), annuloplasty devices (ring annuloplasty, suture annuloplasty). Delivery, and patient selection issues.

In still further alternative embodiments, the various embodiments the surgical access device, device shaft, working channel and balloon visualization module are adapted and configured for use in via transjugular access for use in performing one or more steps of a Plication clip for tricuspid valve procedure to address the shortcomings of or clinical issues as detailed herein. The surgical access device may also include embodiments specific to a transjugular access medical procedure. Additionally or optionally, there may be other modifications of improvements related to improved visualization through application the BVM as well as instruments or devices provided utilizing the hybrid working channel so as to provide a platform for access and direct visualization to guide instruments for use in these procedures. Right ventricular reshaping or tether implantation

### Current State of the Art

Tether is a new, minimally invasive, implantable system for mitral valve regurgitation, developed by Argo Cardiovascular. It is a simple, easy-to-use, mechanical medical device system. It may be applied to the right ventricle for reshaping in RV failure and/or severe tricuspid regurgitation. Additional details are found in published US Patent Application Publication No. US 2020/0268514 entitled, Mechanically Locking Adjustable Cardiac Catheter filed as application Ser. No. 16/789,250, incorporated herein by reference in its entirety.

### Clinical Issues to Solve

The tethers may be placed via a hybrid procedure, both using transcatheter and minimally invasive surgical access.

In still further alternative embodiments, the various embodiments the surgical access device, device shaft, working channel and balloon visualization module are adapted and configured for use in via transjugular access for use in performing one or more steps of right ventricular reshaping or tether implantation procedure to address the shortcomings of or clinical issues as detailed herein. The surgical access device may also include embodiments specific to a transjugular access medical procedure. Additionally or optionally, there may be other modifications of improvements related to improved visualization through application the BVM as well as instruments or devices provided utilizing the hybrid working channel so as to provide a platform for access and direct visualization to guide instruments for use in these procedures.

### Atrial Septal Defect/Persistent Foramen Ovale Closure

### Current State of the Art

The atrial septal defects (ASDs) are the second most common congenital heart defects, which is a birth defect of the heart. ASD is a hole in the wall (septum) that divides the upper chambers of the heart (the most common type of ASD is located at the fossa ovalis). PFO is persistent foramen ovale, a frequent cause of paradoxical emboli, thus, strokes.

### Clinical Issues to Solve

The treatment is the transcatheter closure. It is a safe procedure, but not risk free (serious complications such as erosion and device embolization occur). It is necessary to know the ASD morphology by Transthoracic echocardiogram (TTE) or transesophageal echocardiogram (TEE), or intracardiac echocardiography (ICE).

In still further alternative embodiments, the various embodiments the surgical access device, device shaft, working channel and balloon visualization module are adapted and configured for use in via transjugular access for use in performing one or more steps of an atrial septal defect/persistent foramen ovale closure procedure to address the shortcomings of or clinical issues as detailed herein. The surgical access device may also include embodiments specific to a transjugular access medical procedure. Additionally or optionally, there may be other modifications of improvements related to improved visualization through application the BVM as well as instruments or devices provided utilizing the hybrid working channel so as to provide a platform for access and direct visualization to guide instruments for use in these procedures

### Balloon Atrial Septostomy

### Current State of the Art

The balloon atrial septostomy is a minimally invasive procedure, when a balloon catheter is used to enlarge the foramen ovale (the hole between the left and right atrium). This allows blood from both sides of the heart to mix together in the case of special congenital cardiac abnormalities.

### Clinical Issues to Solve

The deflated balloon is delivered through the foramen ovale to the left atrium, then inflated and pulled back to into the right atrium. The procedure is visualized by echocardiography and hemodynamic monitoring.

In still further alternative embodiments, the various embodiments the surgical access device, device shaft, working channel and balloon visualization module are adapted and configured for use in via transjugular access for use in performing one or more steps of a balloon atrial septostomy procedure to address the shortcomings of or clinical issues as detailed herein. The surgical access device may also include embodiments specific to a transjugular access medical procedure. Additionally or optionally, there may be other modifications of improvements related to improved visualization through application the BVM as well as instruments or devices provided utilizing the hybrid working channel so as to provide a platform for access and direct visualization to guide instruments for use in these procedures.

In still further alternative embodiments, the various embodiments the surgical access device, device shaft, working channel and balloon visualization module are adapted and configured for use in performing one or more steps or a portion of any of the procedures described in the publications listed below. Additionally or optionally, there may be other modifications of improvements related to improved visualization through application the BVM as well as instruments or devices provided utilizing the hybrid working channel so as to provide a platform for access and direct visualization to guide instruments for use in these procedures.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The balloon of the balloon visualization module encloses the camera and light source. Advantageously, the balloon may also serve as a compliant, atraumatic tissue contacting surface. In some exemplary clinical procedures, embodiments of the surgical access device may be adapted and configured especially for electrophysiology mapping and ablation procedures. These embodiments would attach to a surface of the balloon electrodes of a design typical to electrophysiology mapping electrodes. In one implementation, a surgical access device so configured would enable use for simultaneous mapping and ablation procedures. In this use case, the electrodes on a balloon surface would be used for mapping while an ablation electrode is introduced through the working channel of the surgical access device is used for ablation. In an additional aspect, the visual image obtained from the balloon visualization module can be enhanced with the results of the mapping via software. Additionally, visualization of nerve network, junctions and ablation targets by projecting them onto the optical image provided by the balloon visualization module of the surgical access device would be highly beneficial for a variety of electrophysiological applications. In still further embodiments, the electrode pattern on the balloon surface would have the additional benefit of visually marking the balloon surface. Advantageously, this feature can be used to measure distances, diameters, sizes of cardiac structures and features etc. in front of the optical unit of the balloon visualization module.

The balloon of the balloon visualization module may also be adapted and configured as a balloon tamponade device in biopsy procedures and other procedures that risk perforating the heart, causing pericardial effusion and tamponade. Advantageously, the balloon of the balloon visualization module can be used to block the perforation from the inside of the heart, until pericardiocentesis or surgical response can be organized and surgery executed. Furthermore, puncture closing solutions, for example those akin to various vascular closure devices, can be introduced through the surgical access device working channel, and used to block the perforation, deploying such closure devices or plugs from the endocardial surface of the heart towards the pericardial space. Such uses may be sufficient to entirely stop the bleeding through the perforation or can reduce the rate of bleeding and thereby allow more time for emergency surgery.

It is to be appreciated from these additional various uses that the balloon of the surgical access device often plays a multifaceted role beyond its role to exclude blood in front of the optical unit. In one additional aspect, an operator of the surgical access device can raise, lower, modulate, deflate, inflate and perform other forms of modulation of the pressure inside the balloon or adapt the shape of the balloon by modulating the pressure within the balloon. For example, by lowering the pressure, the balloon becomes softer and more pliable, able to mold around intracardiac structures, providing better exclusion of blood and better visualization. Lower pressure inside the balloon would allow the surgical access device to access intracardiac spaces with lower volumes or tighter orifices. In contrast, higher balloon pressure could be advantageous in mapping applications, where it can ensure better apposition of the balloon borne electrodes or other components suited to the procedure against the endocardium or other targeted tissue. In further aspects, modulation and balloon response to pressure may also include aspects where the overall shape of the balloon has one or more different or modified shapes including shapes that are responsive to pressure characteristics within the balloon. By way of example, a pressure responsive shaped balloon would have a first shape at a first pressure and a second different shape at a different pressure. In still another different aspect, a balloon embodiment may have one or more features or contours that appear or are present and functional in response to pressure modulation signatures. By way of example, a balloon may have a first shape without prominent features while at a second pressure one or more features, contours or surfaces are actuated and available for use at the surgical site in conjunction with the other capabilities of the surgical access device. In some embodiments, the balloon characteristics (i.e., soft, firm, hard) balloon shape, features, contours or surfaces may be reversible and controllable by the operator or responsive to inputs from the operator or surgical system. In one embodiment, balloon pressure is controlled by a pump in communication with the balloon inflation lumen of the surgical access device. The pump can be controlled manually, by a floor pedal, or other controller commonly used to control fluid management systems. The pump can be used to inflate, deflate or modulate the balloon pressure and would have safety functions to prohibit the overinflation of the balloon and to prevent balloon burst, or the inadvertent deflation of the balloon under a baseline pressure required for proper optical visualization.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A surgical access device, comprising:
an elongate body having a working channel; and
a balloon visualization module positioned at the distal end of the elongate body, the balloon visualization module comprising a balloon with a camera module positioned within the balloon, wherein when in a stowed configuration, the camera module is configured to be contained within an outer diameter of an access sheath and when deployed in use with an inflated balloon, the camera module moves radially outwardly relative to a longitudinal axis of the elongate body to a position substantially beyond an outer wall of the elongate body.

2. The surgical access device of claim 1, wherein the balloon visualization module is completely within the working channel when stowed.

3. The surgical access device of claim 1, wherein at least a portion of a distal surface of the balloon forms a contact visualization surface configured to contact target tissue and allow visualization of the target tissue therethrough by the camera module.

4. The surgical access device of claim 3, wherein at least 90% of the distal surface of the balloon is within a field of view of the camera module when the camera module is in a deployed configuration.

5. The surgical access device of claim 1, wherein a distal surface of the balloon is rounded.

6. The surgical access device of claim 1, wherein the balloon comprises an optically clear material.

7. The surgical access device of claim 1, wherein the camera module is spaced 8-13 mm from a distal surface of the balloon.

8. The surgical access device of claim 1, wherein the elongate body comprises a plurality of lumens.

9. The surgical access device of claim 1, wherein the elongate body comprises a fluid lumen.

10. The surgical access device of claim 9, wherein a diameter of the fluid lumen is 0.4-0.6 mm.

11. The surgical access device of claim 1, wherein the elongate body comprises a position tracking sensor.

12. The surgical access device of claim 1, wherein the camera module comprises a connector, wire or cable extending proximally towards a proximal end of the elongate body, to provide data and/or power to the camera module.

13. The surgical access device of claim 1, wherein the camera module comprises one or more lights.

14. The surgical access device of claim 1, wherein the camera module is positioned generally parallel to a longitudinal axis of the elongate body.

15. The surgical access device of claim 1, wherein the camera module is positioned at an angle relative to a longitudinal axis of the elongate body.

16. The surgical access device of claim 1, wherein the balloon visualization module comprises a channel extension coupled to a distal end of the working channel.

17. The surgical access device of claim 16, further comprising a camera mount configured to attach to the channel extension and support the camera module.

18. The surgical access device of claim 16, wherein the channel extension comprises a lumen with a cut out portion.

19. The surgical access device of claim 18, wherein the balloon, when inflated, is configured to complete the lumen in at least part of the cut out portion.

20. The surgical access device of claim 18, wherein the cutout portion comprises 60-90% of a length of the channel extension.

21. The surgical access device of claim 16, wherein the channel extension comprises a flexible tube.

22. The surgical access device of claim 16, wherein the channel extension comprises an optically clear material.

23. The surgical access device of claim 16, wherein the balloon comprises a balloon channel configured to surround the channel extension.

24. The surgical access device of claim 23, wherein a diameter of the balloon channel is 3-3.8 mm.

25. The surgical access device of claim 23, wherein a length of the balloon channel is 16-17.4 mm.

26. The surgical access device of claim 16, wherein the balloon is asymmetric around a longitudinal access of the channel extension.

27. The surgical access device of claim 16, wherein greater than 80% of a volume of the balloon is positioned above the channel extension.

28. The surgical access device of claim 1, wherein the balloon comprises a thickness of less than or equal to 0.1 mm.

29. The surgical access device of claim 1, wherein the balloon comprises a durometer of shore 80-90 A.

30. The surgical access device of claim 1, wherein the elongate body comprises a diameter of 12-16F.
